SEQUENCE LISTING (8 sequences)

SEQ ID NO: 1 - DNA, 19 bp, Artificial
  Synthetic PCR Primer
tcgaagagctgctggatga

SEQ ID NO: 2 - DNA, 19 bp, Artificial
  Synthetic PCR Primer
tatgtacactgaccattaa

SEQ ID NO: 3 - DNA, 19 bp, Artificial
  Synthetic PCR Primer
gagcgtgtgtgacagtatt

SEQ ID NO: 4 - DNA, 19 bp, Artificial
  Synthetic PCR Primer
gaactacccaatcgtatgt

SEQ ID NO: 5 - DNA, 23 bp, Artificial
  Synthetic PCR Primer
ctgacacttttgagcacgtgatc

SEQ ID NO: 6 - DNA, 21 bp, Artificial
  Synthetic PCR Primer
aggctccaaaggcacttgact

SEQ ID NO: 7 - DNA, 22 bp, Artificial
  Synthetic PCR Primer
aacgagactctcggcatgctaa

SEQ ID NO: 8 - DNA, 20 bp, Artificial Sequence
  Synthetic PCR Primer
ccggacatctaagggcatca

(19) United States
(12) Patent Application Publication
(10) Pub. No.: US 2009/0186844 A1
(43) Pub. Date: Jul. 23, 2009

(54) SiRNA DELIVERY INTO MAMMALIAN NERVE CELLS

(76) Inventors: Gordon S. Mitchell, Middleton, WI (US); Tracy Lee Baker-Herman, Cottage Grove, WI (US)

Correspondence Address: QUARLES & BRADY LLP, 33 E. MAIN ST, SUITE 900, P.O. BOX 2113, MADISON, WI, 53701-2113, US

(21) Appl. No.: 11/972,376

(22) Filed: Jan. 10, 2008

Related U.S. Application Data
(63) Continuation of application No. 11/009,797, filed on Dec. 10, 2004, now abandoned.
(60) Provisional application No. 60/529,326, filed on Dec. 12, 2003.

(51) Int. Cl. A61K 31/7105

(52) U.S. Cl. 514/44

(57) ABSTRACT

The present invention relates to methods of affecting expression of a target gene, suitably brain-derived neurotrophic factor (BDNF) or related genes in a nerve cell in the central nervous system of a mammal. The method includes formulating and delivering an siRNA composition to a target site on the mammal to affect expression of the target gene in the nerve cell, wherein the target site is cerebrospinal space or muscle tissue innervated by a nerve cell, to down-regulate the target gene. Also disclosed are kits for use in practicing the novel methods of in vivo siRNA.delivery into target cells and gene regulation.

### CROSS-REFERENCE TO RELATED APPLICATIONS

This application is a continuation of U.S. patent application Ser. No. 11/009,797, filed Dec. 10, 2004 which claimed the benefit of U.S. Provisional Application No. 60/529,326 filed Dec. 12, 2003. Each application is incorporated herein by reference as if set forth in its entirety.

### STATEMENT REGARDING FEDERALLY SPONSORED RESEARCH OR DEVELOPMENT

This invention was made with United States government support awarded by the following agency: NIH Grant Nos. HL65383 and HL 07654. The United States has certain rights in this invention.

### BACKGROUND OF THE INVENTION

Small interfering RNA (siRNA) can induce specific gene knockdown or interference with gene translation to new proteins, commonly known as RNA-interference (RNAi). RNAi has been used to assess physiological functions of specific genes in C. elegans (Fire, et al., (1998) Nature 391, 806-811), Drosophila (Kennerdell, J. R. and Carthew, R. W. (1998) Cell 95, 1017-1026.), zebrafish (Wargelius, et al., (1999) Biochem Biophys Res Commun 263, 156-161) and mice (McCaffrey, et al., (2002) Nature 418, 38-39). The RNAi mechanism of action is generally thought to involve siRNA induction of a protein complex called RNA induced silencing complex (RISC), resulting in sequence specific mRNA cleavage or translational inhibition. RNAi produces sequence-specific reduction of gene expression in various mammalian in vitro systems. Recently, RNAi was shown to suppress the expression and activity of a luciferase transgene in adult mice (McCaffrey, et al., (2002)) suggesting that RNAi may become a valuable molecular and possibly clinical tool for use in vivo. A major advantage of RNAi over a similar protocol, using antisense oligonucleotides, is that the use of antisense oligonucleotides to produce chronic effects requires constant or repetitive delivery of a large molecule that is difficult to transport and maintain in appropriate conformation, is expensive, and is generally impractical for more than a few days.

In contrast, siRNA acts catalytically at sub-molar ratios to cleave up to 95% of the target mRNA in the cell. The RNA interference effect can be long-lasting and may be detectable after many cell divisions. These properties make siRNA extremely effective at inhibiting target gene expression once introduced into the cell. However, a major uncertainty in siRNA research has been the actual in vivo delivery of the siRNA to different target organs.

A primary focus of gene therapy has been based on discovering strategies for delivering genetic material, usually DNA into living cells. In the past, researchers have used viral vectors to efficiently transfect cells in vivo in an attempt to overcome problems with delivering genetic material, such as siRNA However, attempts to induce RNA interference using viral vectors in mammalian cell lines have been met with limited success, due in part to the induction of the interferon response, resulting in a general inhibition of protein synthesis.

More recently, non-viral gene delivery has been used to bypass the potential deleterious immune effects of attempting to reduce gene expression in vivo. Such non-viral methods have included the development of an intravascular delivery method that allows for the efficient delivery of siRNAs or other genetic material to liver cells (Herweijer, et al., (2001) Journal of Gene Medicine 3(3):280-91; and Wolff et al., (1997) Hum Gene Ther 8:1763-72). However, in nearly all instances to date, non-viral DNA/polymer and/or DNA/lipid polyplexes have failed to deliver genes to cells in vivo as effectively as in vitro because the non-viral particles aggregate in physiologic solutions and the large size of the aggregates interferes with their ability to remain in the blood to access target cells. In addition, previously-developed non-viral particles required a net positive charge in order for the packaged DNA to be fully protected, preventing their contact with target cells in vivo.

Wolff et al., have also disclosed that intravascular siRNA delivery using a catheter mediated intravenous gene delivery to hepatocytes is effective to knock down target gene expression, using a polymer-based gene delivery system (see U.S. Pat. No. 6,265,387).

In addition, Makimura, et al., disclosed that when siRNA was delivered in the hypothalamus of rats using a plasmid-based system, metabolic function could be influenced. It is believed that the plasmid-based method lead to the transcription of a short double stranded RNA product with a hair-pin loop in vivo, resulting in reduction of target gene mRNA levels. Their findings suggest that RNAi protocols can be used to decrease specific endogenous hypothalamic protein levels in the central nervous system leading to an increase in the metabolic rate and a decrease in body weight (Makimura, et al. (2002) BMC Neurosci 3, 18).

However, the applicability of RNAi to alter functions in other regions of the nervous system of mammals has not yet been demonstrated. One reason is that effective delivery has posed a particular uncertainty with respect to the organs related to the central nervous system because other neural structures (unlike the hypothalamus) are inaccessible to the blood due to the presence of the blood brain barrier. In the blood, siRNA and miRNA (small regulatory RNA molecules, referred to as microRNAs) are quickly degraded, thereby limiting blood delivery of small RNAs as a therapeutic tool to prevent translation of, or to knock down, endogenous mRNA.

In fact, the inability to effectively utilize siRNA to regulate gene expression in the nervous system was recently described by Isacson et al. They described studies where intrastriatal infusions of siRNA targeted to dopamine D1 receptor mRNA did not reduce dopamine D1 receptor mRNA levels or protein levels in intact rats. These results were contrary to in vitro observations where a 76% reduction in dopamine D1 receptor ligand binding was obtained. Accordingly, Isacson et al., concluded that synthetic siRNA, administered by direct infusion into rat brain, was not capable of inducing RNA interference (Isacson, et al., (2003) Acta Physiol Scand, 179, 173-177).

Accordingly, it would be desirable to develop novel and effective methods for targeting and inhibiting protein function in vivo by use of RNA interference because thus far a major limitation has been the difficulty in effectively delivering the siRNAs or miRNAs to the targeted cells in the central nervous system.

### BRIEF SUMMARY OF THE INVENTION

The present invention is summarized as methods for affecting gene expression of a specific target gene in specific types of nerve cells in the central nervous system of a mammal. The methods include formulating an siRNA composition constructed to have a strand complementary to a portion of the target gene; and delivering the siRNA composition to a target site on the mammal to affect expression of the target gene in the nerve cell. The target injection site is a muscle tissue innervated by motor nerve cells or by localized injections within the cerebrospinal fluid, such that the expression of the target gene may be down-regulated.

In one aspect of the invention the target gene is brain derived neurotrophic factor (BDNF).

In another aspect, the target gene is a receptor or a signaling molecule, such as MAP kinases or phosphatases regulating the expression of BDNF. In this aspect, the nerve cell is a motoneuron that sends processes from the cell body in the medulla or spinal cord to a target muscle, or sensory neurons that send processes to the muscle or skin.

In another aspect, the muscle tissue is a tongue or a diaphragm muscle.

In another aspect, the siRNA composition is delivered to the target site in the presence of a delivery reagent, preferably Oligofectamine™.

In another aspect the invention also provides a kit for use in affecting gene expression of a target gene in a nerve cell in the central nervous system of a mammal. The kit includes the siRNA composition; and instructions for practicing the method of affecting gene expression.

In another aspect, the invention provides insight into the regulatory mechanisms of BDNF, MAP kinases and protein phosphatases in neuronal responses to intermittent hypoxia (and possibly a form of neuroplasticity in the control of breathing, known as respiratory long-term facilitation or LTF). Also provided is the utility of using siRNA technology directed to BDNF or protein phosphatases to regulate gene function in the medulla and spinal cord in vivo. It is also encompassed that the invention may be extended to other molecules and will be useful in the treatment of a variety of medical conditions. Accordingly, in another embodiment, genes in combination with BDNF or alone are targeted which could influence motoneuron function, such as for example, receptors and signaling molecules downstream of BDNF or that act in parallel with BDNF or that may regulate BDNF.

In still another embodiment, the various modes of delivering the siRNA composition may prove highly useful in treating motoneuron related conditions, such as, for example, obstructive sleep apnea, spinal cord injury, degenerative motoneuron disease (ALS), polio. A similar approach, with siRNA administration to the skin or nerve(s) may be useful in treating chronic pain. Other applications where the methods of the invention may be useful are described in detail below.

Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used.

Other objects, advantages and features of the present invention will become apparent from the following specification taken in conjunction with the accompanying drawings.

### BRIEF DESCRIPTION OF THE SEVERAL VIEWS OF THE DRAWINGS

FIG. 1A-B illustrates that intermittent hypoxia elicits a form of phrenic motoneuron plasticity known as pLTF and increased BDNF synthesis in ventral spinal segments associated with the phrenic motor nucleus; (A) shows a representative raw and integrated phrenic neurogram taken before and 60 min following three, 5 min episodes of hypoxia (11% O₂). In this example, phrenic amplitude is increased 80% above baseline levels 60 min post-hypoxia, indicating pLTF; and (B) 60 min following three, 5 min episodes of hypoxia, BDNF concentration increased in ventral spinal segments C₃-C₅ when compared to controls not receiving hypoxia. *significantly increased, (P<0.05); data are mean±SEM.

FIG. 2A-C graphically illustrates the regulation of ventral cervical BDNF following intermittent hypoxia; (A) shows the BDNF concentration expressed as a percentage increase from corresponding controls in rats pretreated with intrathecal artificial CSF, methysergide or emetine prior to hypoxia. Rats with intrathecal artificial CSF injections had a significant increase in ventral C₃-C₅ BDNF concentration 60 min following intermittent hypoxia. Intrathecal methysergide or emetine blocked BDNF increases 60 min post-intermittent hypoxia, suggesting a serotonin-dependent increase in BDNF synthesis; (B) shows BDNF concentration expressed as a percentage increase from controls in rats exposed to intermittent hypoxia (CSNX-sham), intermittent hypoxia+carotid denervation (CSNX), or intermittent hypercapnia; and (C) shows the correlation between average changes in BDNF (% control) in ventral cervical segments with average pLTF (% baseline) 60 min post-intermittent hypoxia or hypercapnia. Groups are: intermittent hypoxia with artificial CSF (), intermittent hypoxia without intrathecal injections (φ), intermittent hypoxia with intrathecal methysergide (▪), intermittent hypoxia with intrathecal emetine (▾), intermittent hypoxia in CSNX-sham (▴), intermittent hypoxia with CSNX (▴), intermittent hypercapnia (♦) and intermittent hypoxia with siRNA (m). Average pLTF at 60 min post-hypoxia is strongly correlated with percentage increases in BDNF (R²=0.67, P=0.01). *significantly increased, (P<0.05); data are mean±SEM.

FIG. 3A-C shows that intrathecal BDNF facilitates phrenic motor output; (A) shows a representative trace of integrated phrenic discharge before, during and 90 min following intrathecal BDNF (100 ng) injections; (B) shows the average data depicting the percentage change in phrenic burst amplitude 30, 60 and 90 min following intrathecal BDNF (e), vehicle (artificial CSF+0.1% BSA; ▪) or BDNF+K252a (A); and (C) shows that intrathecal BDNF did not affect XII activity, suggesting a spinal site of action. *significantly increased from baseline and significantly different from other groups (P<0.05); data are mean±SEM.

FIG. 4A-B shows that BDNF siRNA reduced BDNF mRNA in vitro and hypoxia-induced BDNF synthesis in vivo; (A) BDNF mRNA is reduced 75% from control 24 hours post-transfection in HT-22 cells; (B) shows no significant change in baseline BDNF protein levels in ventral gray matter (C₄-C₅) 3 hours following intrathecal BDNF siRNA injections in adult rats. However, the increase in BDNF concentration normally observed following intermittent hypoxia is blocked by the prior administration of BDNF siRNA. *significantly different from baseline, #significantly different groups (P<0.05); data are mean±SEM.

FIG. 5A-C illustrates that BDNF siRNA and Trk receptor inhibition with K252a block pLTF; (A) shows representative integrated phrenic neurograms illustrating the development of pLTF during and following three, 5 min hypoxic episodes; (B) shows average data illustrating that intrathecal BDNF (but not scrambled) siRNA pretreatment blocked pLTF 60 min post-intermittent hypoxia (% change from baseline activity); and (C) shows average data illustrating that intrathecal K-252a pretreatment attenuated pLTF 60 min post-intermittent hypoxia (% change from baseline activity), when compared to rats receiving vehicle injections. *significantly increased from baseline and significantly different from other treatment groups (P<0.05); data are mean±SEM.

FIG. 6 is a graph showing that an injection having a BDNF siRNA composition into the tongue muscle can inhibit LTF in hypoglossal nerve activity while leaving LTF in the phrenic nerve intact. This experiment shows that intramuscular injections can influence the function of discrete motoneuron populations 3 days after an intramuscular siRNA injection, expressed here as a block in hypoglossal (but not phrenic) LTF.

FIG. 7 shows that diaphragm injections of BDNF siRNA abolish phrenic (but not XII) LTF. Black bars represent average phrenic and XII LTF 60 min post-intermittent hypoxia in anesthetized rats. Gray bars represent phrenic and XII nerve activity (% baseline) 60 min following intermittent hypoxia in one rat that had received a diaphragm injection of BDNF siRNA 3 days prior to hypoxic exposures. Thus, BDNF within phrenic motoneurons may be necessary for phrenic LTF. * indicates significant pLTF (p<0.05). This experiment is the complement of FIG. 6 and shows that phrenic motoneurons can be targeted with intradiaphragm injections of siRNA independently from hypoglossal (tongue) motoneurons. Further, this experiment supports the contention that intramuscular injections of siRNA effectively influence the behavior of specific populations of nerve cells and thus may be used to target gene therapy by use of RNA interference.

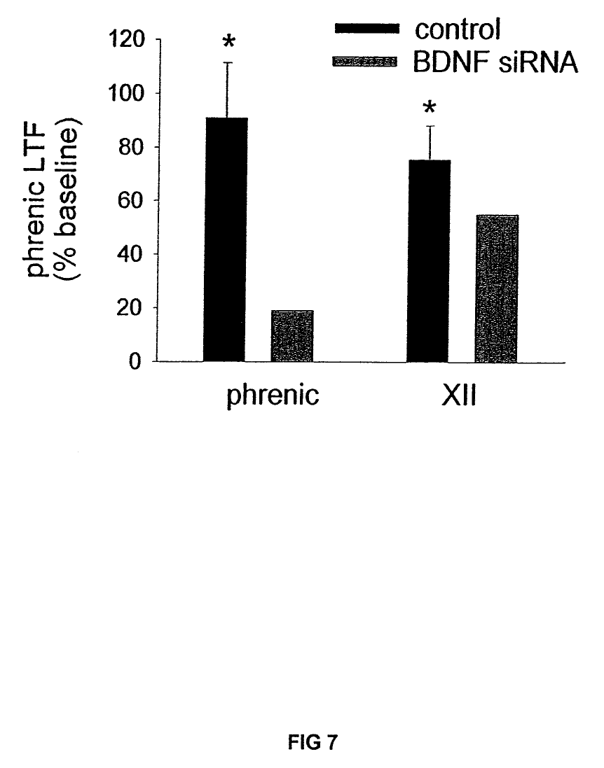

FIG. 8A-B illustrates the underlying idea of the invention, where a differential balance of kinase and phosphatase activation accounts for differences between (A) intermittent and (B) sustained hypoxia in their capacity to elicit pLTF. During sustained hypoxia, phosphatase activation halts the mechanism leading to pLTF. By targeting kinase or phosphatase mRNAs with siRNAs with a complementary sequence, the expression of BDNF and neuroplasticity induced by BDNF can be regulated.

FIG. 9 shows intermittent (but not sustained) hypoxia increases BDNF in ventral cervical gray matter. Specifically, BDNF protein concentration increases in ventral C4-C5 60 min following intermittent, but not sustained, hypoxia in unanesthetized rats (n=4 per group). * indicates significantly greater than control (p<0.05).

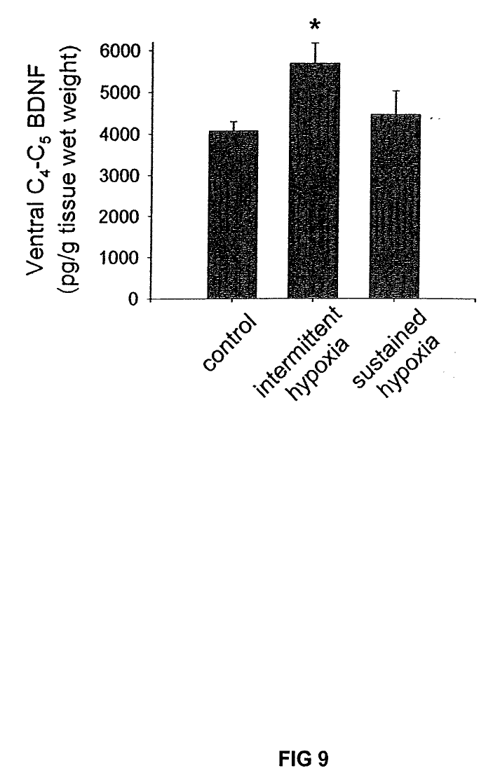

FIG. 10A-B shows intermittent (but not sustained) hypoxia elicits persistent activation of ERK1/2 MAP kinases. (A) immunoblot of phosphorylated (activated) ERK1/2 MAP kinase in ventral C4-C5 gray matter in rats exposed to normoxia (control), intermittent hypoxia (1H) or sustained hypoxia (SH). Tissues were harvested 60 min post-hypoxia. Also, shown is that (B) intermittent (but not sustained) hypoxia elicits persistent ERK1/2 MAP kinase activation in spinal regions associated with the phrenic motor nucleus. Average density of phospho-ERK1/2 MAP kinase in ventral C4-C5 60 min post-intermittent or sustained hypoxia in awake rats (n=4, each), normalized to total ERK1/2 MAP kinase and expressed as a percentage change from rats exposed to normoxia. * significantly increased and different from sustained hypoxia (p<0.05).

FIG. 11 shows ERK1/2 MAP kinase activation is BDNF-dependent. Specifically, shown is an immunoblot for enzymatically activated (phosphorylated) ERK1/2 MAP kinase demonstrating that BDNF synthesis is necessary for intermittent hypoxia (1H) induced ERK1/2 activation near phrenic motoneurons. BDNF siRNA was injected over C4 1.5 hours prior to intermittent hypoxia. Ventral gray C4-C5 was collected 60 min post-hypoxia. The control rat received BDNF siRNA for an equivalent duration, but without hypoxia. This experiment demonstrates that BDNF siRNA administration can regulate the activation of downstream signaling molecules.

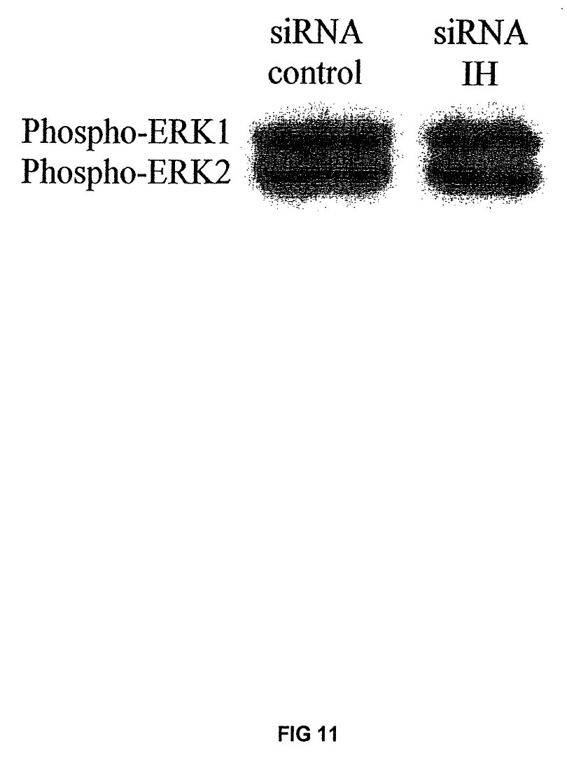

FIG. 12 shows sustained (but not intermittent) hypoxia increases protein phosphatase 2 (PP2) activity. Awake rats were exposed to normoxia, intermittent hypoxia (1H) or sustained hypoxia (SH) (n=1 per treatment). 15 min post-hypoxia, ventral C4-C5 was harvested and assayed for protein phosphatase-2 activity. The rat exposed to sustained hypoxia had increased PP2 activity compared to rats given normoxia or intermittent hypoxia. Each sample was assayed in triplicate; error bars represent standard deviation of triplicates.

FIG. 13 shows that sustained hypoxia elicits phrenic LTF following protein phosphatase inhibition. Intraspinal inhibition of protein phosphatases with okadaic acid reveals pLTF in a rat exposed to sustained hypoxia (SH+okadaic acid). In other rats, intermittent (1H) (but not sustained, SH) hypoxia elicits pLTF 60 min post-hypoxia. * indicates response significantly lower than IH (p<0.05). This experiment suggests that, by targeting protein phosphatases with siRNAs, molecules that regulate the expression of BDNF and other downstream molecules may be influenced. Thus, by targeting and degrading protein phosphatases, BDNF function can be enhanced.

### DETAILED DESCRIPTION OF THE INVENTION

The present invention generally relates to methods of affecting gene expression of a target gene in a nerve cell in the central nervous system of a mammal. The method includes formulating an siRNA composition constructed to have a strand complementary to a portion of the target gene; and delivering the siRNA composition to a target site in the mammal to affect gene expression of the target gene in the nerve cell. The target site may be a muscle tissue linked by nerve cell(s) or cerebrospinal space, such that the target gene or its ability to produce new protein is down-regulated in the nerve cell.

In accordance with the invention, one embodiment provides that direct delivery of the siRNA composition into the intrathecal space of a mammal, effectively interfered with BDNF mRNA and blocked increases in BDNF in the cervical spinal cord elicited by a reduced flow of oxygen called intermittent hypoxia. Intermittent hypoxia causes a form of serotonin-dependent spinal synaptic plasticity known as phrenic long-term facilitation (pLTF).

In this embodiment, a target gene, preferably BDNF, in a nerve cell in the central nervous system of a mammal was down-regulated. Although, it is envisioned that a receptor or a signaling molecule downstream of BDNF, or acting in concert with BDNF, or regulating BDNF, may be targeted as well. The method includes formulating an siRNA composition constructed to have a strand complementary to a portion of BDNF mRNA; and delivering the siRNA composition to a target site in the mammal to cause down-regulation of BDNF in the motor nerve cell. The target neurons are motoneurons in the medulla or spinal cord. The siRNA delivery site is the muscle linked to the motoneurons or the cerebrospinal space. Applicants note that an exemplary delivery reagent is a cationic lipid transfection reagent, such as Oligofectamine™ used to deliver the siRNA composition to the target site. It is also noted that the nerve cells of the central nervous system are important for normal respiratory function and must provide an appropriate motor output, triggering respiration.

Furthermore, another embodiment provides that indirect delivery of the siRNA composition intramuscularly into muscles innervated by motoneurons protected siRNA molecules from circulating RNAses in the blood and resulted in transport of intact siRNA molecules back to the nerve cells. This indirect transport of siRNA molecules from the muscles to the nerve cells resulted in blocking LTF following intermittent hypoxia in the hypoglossal nerve (the motor nerve of the tongue) but not phrenic nerve (associated with the diaphragm) motor output, when the siRNA was injected into the tongue muscle. The effect on motoneurons that attach to the tongue demonstrates specific delivery to the targeted motoneurons located in the medulla. When the siRNA was injected into the diaphragm instead of the tongue, LTF was blocked in the phrenic motor output, but not the hypoglossal motor output. The effect on motoneurons that attach to the diaphragm demonstrates specific delivery to the targeted motoneurons located in the spinal cord. Thus, the methods of the present invention are able to affect target gene expression and produce physiological affects by delivering siRNA molecules directly or indirectly into a target site.

Specifically, this embodiment discloses a method of down-regulating a target gene, in a nerve cell in the central nervous system of a mammal. Again, an exemplary target gene is brain derived neurotrophic factor (BDNF); however, a receptor or a signaling molecule downstream of BDNF, or a molecule acting in concert with BDNF, or a molecule that regulates the expression of BDNF is also encompassed by the invention. The method includes formulating an siRNA composition constructed to have a strand complementary to a portion of BDNF; and delivering the siRNA composition to a target site on the mammal to cause down-regulation of BDNF in the nerve cell, wherein the target site is a muscle tissue linked to the nerve cell. Preferably the muscle tissue is a tongue or a diaphragm muscle. Other muscles and other motoneurons involved with motor functions other than breathing are encompassed by the invention. Also, an exemplary delivery reagent is a cationic lipid transfection reagent, such as Oligofectamine™ used to deliver the siRNA composition to the target site.

Also encompassed within the scope of the invention are the use of other types of therapies to supplement the methods of the invention, under conditions suitable for affecting gene expression to treat the medical condition. Other envisioned therapies include those known in the art, such as monoclonal antibody therapy, chemotherapy, radiation therapy, trophic factor supplementation, and analgesic therapy, or a combination thereof.

The invention also provides for kits that would include components to be used in practicing the methods of in vivo siRNA molecule delivery to a target cell, such as a nerve cell in the nervous system of a mammal to affect cellular gene expression. The subject kits may generally include siRNA molecules, as described herein, alone or complexed with a delivery reagent (siRNA composition) for delivery into the target cell. The subject kits may further include an aqueous delivery vehicle, e.g. a buffered saline solution, etc. In addition, the kits may include a competitor RNA, for competing with the target gene. In the subject kits, the above components may be combined into a single aqueous composition for delivery into the host or separate as different or disparate compositions, e.g. in separate containers. Optionally, the kit may further include an intrathecal or intramuscular delivery means for delivering the aqueous composition to the host, e.g. a syringe etc., where the delivery means may or may not be pre-loaded with the aqueous composition.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g. a piece or pieces of paper on which the information is printed, in the packaging of the kit, in a package insert, etc. Yet another means would be a computer readable medium, e.g. diskette, CD, etc., on which the information has been recorded. Yet another means that may be present is a website address which may be used via the internet to access the information at a removed site. Any convenient means may be present in the kits.

### DEFINITIONS

The term “hypoxia” as used herein refers to a state of oxygen deficiency in the body which is sufficient to cause an impairment of function. In general, hypoxia is the reduction in partial pressure of oxygen within the blood caused by inadequate oxygen transport, such as a period without breathing or a reduction in the amount of oxygen in the air, such as at altitude.

As described by the invention, there are two types of hypoxia: intermittent hypoxia and sustained hypoxia. Intermittent hypoxia is broadly defined as repeated episodes of hypoxia interspersed with episodes of normal oxygen. Intermittent hypoxia is caused by a frequent occurrence as exemplified by lung diseases and sleep-disordered breathing. Intermittent hypoxia triggers a cascade of physiologic and biologic intraneuronal events that are triggered by O₂ deprivation and that can lead to adaptation and survival or neuronal damage. Specifically, it causes a form of serotonin-dependent spinal synaptic plasticity known as respiratory long-term facilitation (LTF). Sustained hypoxia is triggered by long-term sojourns at high altitude and chronic lung disease.

It is noted that although, the cellular mechanisms that differentiate intermittent and sustained hypoxia in the context of LTF are not yet understood, applicants believe that based on the results presented herein the detailed description that intermittent and sustained stimulation (hypoxia/serotonin) exert differential effects on protein phosphatases, and that these differences account for the pattern sensitive expression of LTF.

The term “long-term facilitation” or “LTF” as used herein refers to widely studied model of respiratory plasticity. LTF was first described by Millhorn and colleagues more than two decades ago. They observed that integrated phrenic nerve activity remained elevated above pre-stimulation levels for at least 90 min following episodic stimulation of chemoafferent neurons in the carotid sinus nerve in anesthetized, paralyzed and ventilated cats. Even though the concept of LTF has been known for some time, researchers are just beginning to understand the detailed cellular and synaptic mechanisms giving rise to this form of neuroplasticity. In accordance with the methods of the invention, applicants have utilized anesthetized, paralyzed and ventilated rats as a model to study mechanisms of respiratory LTF in vivo. LTF is observed in several respiratory-related nerves, such as the phrenic (diaphragm innervation) and hypoglossal (tongue innervation), and is primarily revealed as an enhancement of nerve burst amplitude that develops 15-30 min post-episodic hypoxia, and lasts for more than 1 hour.

More specifically, the term “phrenic LTF” or “pLTF” arises from a central neural mechanism, largely within or near phrenic motoneurons. Through the years applicants have found that pLTF is generally due to a central mechanism since it can be elicited by electrical stimulation of carotid chemoafferent neurons in the absence of hypoxia and is not observed in carotid chemoafferent activity following intermittent hypoxia; is observed in short-latency spinally evoked responses in phrenic motor output and is blocked by spinal application of serotonin receptor antagonists, protein synthesis inhibitors and by small interfering RNAs (siRNAs) directed against BDNF mRNA.

Furthermore, applicants have found that pLTF requires spinal serotonin 5-HT2A receptor activation for its initiation, but not for its maintenance. Since pLTF requires spinal serotonin-dependent protein synthesis as early as 15 min post-intermittent hypoxia. It is believed that that intermittent 5-HT2A receptor activation on phrenic motoneuron dendrites initiates the synthesis of new proteins via translation of existing, dendritic mRNA (i.e., increased BDNF synthesis and pLTF are translation-dependent, but transcription-independent.)

One protein that is critically involved in pLTF is brain derived neurotrophic factor (BDNF). Intermittent hypoxia elicits serotonin-dependent increases in the synthesis of BDNF in ventral spinal segments encompassing the phrenic motor nucleus. As described herein applicants have used RNA interference with small, interfering RNAs (siRNA) to block spinal BDNF synthesis following intermittent hypoxia, which abolished pLTF and demonstrated that new BDNF synthesis is necessary for its underlying mechanism. Also, inhibition of the high affinity BDNF receptor (TrkB) abolishes pLTF, whereas spinal BDNF applications facilitate phrenic burst amplitude similarly to pLTF.

The term “siRNA,” “siRNA duplex” or “siRNA molecule” as used herein refers to a (duplex) double stranded nucleic acid molecule capable of RNA interference “RNAi”, see for example Elbashir et al., (2001), Nature, 411, 494-498. As used herein, siRNA molecules need not be limited to those molecules containing only native or endogenous RNA nucleotides, but further encompass chemically modified nucleotides and non-nucleotides. An example, of a suitable modification is siSTABLE™ siRNA (available through Dharmacon Research Inc.) siSTABLE™ siRNA is a proprietary form of siRNA in which the complementary strands have been chemically modified to enhance duplex stability, silencing longevity and potency, without increasing cellular toxicity. siSTABLE™ siRNA also includes modifications that also inactivate the sense strand of the duplex, eliminating its potential participation in off-target silencing.

The siRNA duplexes of the invention, described in detail below were designed and synthesized by Dharmacon. However, specific siRNA's, which can be designed and manufactured, are available through Oligo Engine (Seattle, Wash.), Ambion (Austin, Tex.) or SiRNA Technologies among others.

The term “siRNA composition” or “siRNA duplex pool” as used herein refers to naked siRNA molecules associated with a transfection, delivery reagent. The siRNA composition is delivered to a target site to down-regulate expression of a target gene or impair its ability to translate new protein through RNA interference.

A “delivery reagent” as used herein is a compound or compounds used in the prior art that bind(s) to or complex(es) with polynucleotides, preferably siRNA molecules and mediates their entry into cells. The delivery reagent also mediates the binding and internalization of siRNA into cells.

It is noted that the siRNA molecules of the invention may be added directly, complexed with cationic lipids, packaged within a delivery reagent, or otherwise delivered to target cells or tissues under conditions suitable for administration. Examples of delivery reagents include cationic liposomes and lipids, calcium phosphate precipitates, rechargeable particles and polylysine complexes. Typically, the delivery reagent has a net positive charge that binds to the siRNA's negative charge. The transfection reagent mediates binding of siRNA to a cell via its positive charge (that binds to the cell membrane's negative charge) or via ligands that bind to receptors in the cell. For example, cationic liposomes or polylysine complexes have net positive charges that enable them to bind to DNA. Other delivery reagents used to transfer genes into cells that are well known in the art are also encompassed within the scope of the invention. These include complexing the polynucleotides on particles that are then accelerated into the cell or elecroporation. The charge increases the permeability of the cell.

A preferred transfection reagent of the invention is Oligofectamine™ (Invitrogen; Carlsbad, Calif.). Other suitable commercially available transfection reagents include, for example, Lipofectin™ 2000 (Invitrogen Corp.; Carlsbad, Calif.) or TransIT-TKO® Transfection Reagent from Mirus (Madison, Wis.). Also encompassed within the scope of the invention is the local administration of siRNA complexes to relevant tissues ex vivo or in vivo through injection or infusion pump, with or without their incorporation in biopolymers.

Furthermore, it is encompassed that the delivery of siRNA compositions can be achieved through a variety of different modes of administration. Exemplary methods of delivering siRNA compositions of the invention include intrathecal or intramuscular injections.

The term “nerve cell” as used herein refers to a cell in the central nervous system which preferably includes the brain and spinal cord, but may also include peripheral nerves and ganglia. Multiple cell types may be included in this description, including neurons, astrocyte glial cells, microglial cells, oligodendrocytes, Schwann cells, and epithelial cells of the choroid plexus.

The term “target gene” as used herein refers to a polynucleotide, preferably an mRNA which has a portion of its polynucleotide sequence complementary to an siRNA molecule of the invention. Both cytoplasmic and nuclear target genes are encompassed by the invention. A preferred target gene may include BDNF and any receptors or signaling molecules downstream of BDNF, or regulating BDNF, or acting in concert with BDNF.

Applicants believe that siRNA compositions targeted against the BDNF gene, may be applicable to other mRNA targets that can influence motoneuron function. For example, BDNF gene expression can be regulated by targeting receptors and signaling molecules downstream of BDNF, or molecules that regulate the expression of BDNF such as transcription factors (i.e., calcium response element (CRE), nuclear factor of activated T-cells isoform 4 (NFATc4), among others) or protein phosphatases (described below). Major downstream targets of BDNF include the mitogen-activated protein (MAP) kinases ERK-1 and ERK-2, kinases that regulate ERK activation (MEKK), the high affinity tyrosine kinase receptor for BDNF (TrkB), protein kinase C, protein kinase A and CAMKII. Since, many of these molecules are kinases that are in turn controlled by kinases, applicants believe that by targeting phosphatases that deactivate them, the balance could shift in the direction of net system activation. Thus, other cellular targets of siRNAs may include protein phosphatases.

Among the phosphatases, protein phosphatases 1, 2A and 2B are the strongest candidates for a prominent role in pLTF and the possibility of enhancing respiratory motoneuron function. Of these, protein phosphatase 2A (PP2) is preferred as detailed below in the examples, where sustained hypoxia was found to preferentially activate PP2, which acts as an endogenous “brake” and prevents the expression of pLTF by halting its fundamental mechanism at the level of kinase activation.

One of the major classes of PP2A substrates is the serine-threonine protein kinases, and PP2A is sometimes regarded as a kinase phosphatase. For example, PP2A inhibits protein kinase C (PKC) activation and there is a direct physical association between PP2A and PKC in mammalian cells. Indeed, prolonged activation of PKC causes its own dephosphorylation and the subsequent down-regulation of its own activity under the influence of PP2A. PP2A also inactivates ERK 1/2 MAP kinases.

Other potential cellular targets include NOGO (an axon growth inhibitor in the adult CNS), myelin basic protein, serotonergic receptors, GABA receptors, glutamate receptors (and their subunits) and/or specific potassium and chloride channels. Specific targets can be chosen to promote plasticity and/or survival of motoneurons. By targeting siRNA compositions to signaling molecules as noted above, the delivery approaches described by the invention may prove highly useful as a therapy for a variety of conditions.

The term “BDNF” as used herein refers to brain-derived neurotrophic factor (see Genbank Accession number: BDNF, NM_{—}012513.) Neurotrophins such as BDNF and neurotrophin-3 (NT-3) play key roles in many forms of neuroplasticity (8-12). Applicants believe that BDNF is produced in the following relevant signaling cascade: 5-HT_{2A} receptors activate a G protein (Gα_{q}), phospholipase C and then protein kinase C (PKC). PKC may then lead to new BDNF synthesis via (direct or indirect) phosphorylation of relevant translation initiation factors, such as the eukaryotic initiation factor 4E (eIF-4E). Subsequently, BDNF is released from the dendrites of phrenic motoneurons, and may act pre- and/or post-synaptically by activating the high affinity receptor tyrosine kinase (TrkB). After BDNF elicits autocrine activation of TrkB receptors on phrenic motoneurons, signal transduction cascades are activated that establish pLTF, at least initially. Although cellular mechanisms downstream from BDNF are still unknown, a likely contributor is the extracellular regulated kinases 1/2 (ERK1/2), members of the mitogen-activated protein kinase family (MAP kinase). For example, it is believed that exogenous BDNF activates ERK1/2 MAP kinase in multiple neuron types, including spinal motoneurons. ERK1/2 activation is required for many forms of synaptic plasticity, including hippocampal LTP and long-term synaptic facilitation in Aplysia.

Therefore, in accordance with the invention, BDNF synthesis is necessary for pLTF since interference with BDNF mRNA translation with small interfering RNAs (siRNA) can abolish pLTF. It is believed that BDNF likely induces pLTF via the high affinity TrkB receptor since inhibition of receptor tyrosine kinases abolished pLTF. The results presented herein also illustrate that BDNF is necessary and sufficient for pLTF following intermittent hypoxia.

By “down-regulate”, “inhibit” or knock-down” it is meant that the expression of a gene, or level of RNAs or equivalent RNAs encoding one or more proteins, or activity of one or more proteins is reduced below that observed in the absence of the siRNA composition. In accordance with the invention, BDNF mRNA is the target of down regulation by siRNAs for purposes of blocking BDNF synthesis following intermittent hypoxia (loss of function). Alternatively, protein phosphatase 2A would be a suitable target of down regulation to promote BDNF synthesis following sustained hypoxia (gain of function).

In accordance with the invention, it is believed that therapeutic approaches based on loss of function (minimize spasticity or chronic pain) or gain of function (obstructive sleep apnea or spinal cord injury treatments or als) are within reach. In the loss of function approach, BDN For downstream molecules such as ERK or the activators of ERK and MEKK are suitable gene targets. In the gain of function approach, other molecules that regulate or work in concert with bBDNF to add function are suitable gene targets. Examples of such molecules would be the protein phosphatases. Also, it is envisioned that molecules such as purinergic receptors may be suitable targets, because intermittent hypoxia upregulates important ATP receptors such as the P2X7 receptor.

As used herein the term “plasticity” refers to a change in system behavior based on experience. Such plasticity is suitably exhibited as a property of the neural network underlying respiratory control. Plasticity has many potential roles in guiding development and aging of the respiratory control system. Indeed, the neural elements that control breathing must adapt to a wide range of physiological and/or environmental changes throughout life, such as birth, pregnancy, obesity, respiratory infection, altitude exposure, neural injury, and even the normal deterioration of pulmonary mechanics and gas exchange with aging. Despite the critical importance of respiratory plasticity, particularly during disease, the detailed mechanisms giving rise to plasticity are not well understood. Therefore, in accordance to the invention, pLTF following intermittent and sustained hypoxia is a model of spinal, serotonin-dependent plasticity with great potential to advance the understanding of neurotrophins, their regulation, and their role in neuroplasticity. Such understanding of serotonin-dependent respiratory plasticity may have important implications in the development of therapeutic strategies to respiratory disorders including sudden infant death syndrome (SIDS), obstructive sleep apnea, respiratory insufficiency following spinal cord injury, respiratory insufficiency attendant to neurodegenerative motoneuron diseases (e.g., ALS), infectious motoneuron diseases (e.g., Polio) and other disorders that affect respiratory control (e.g., Rhett Syndrome).

### APPLICABILITY OF THE INVENTION

It is believed that the methods of the invention may be used in affecting gene expression of target genes to treat a variety of medical conditions. For example the methods of the invention may be employed to use siRNA compositions directed against target molecules such as for example, BDNF and PP2A, to down regulate gene expression and facilitate treatment of motoneuron related conditions, such as, obstructive sleep apnea, spinal cord injury, degenerative motoneuron diseases (e.g., ALS and polio). A similar approach may be used by applying siRNA to tissues such as skin and muscle, decreasing the expression of for example, BDNF and associated molecules to minimize chronic pain. This approach to minimize chronic pain will not be through effects on motoneurons, but via actions on sensory nerve cells. With respect to chronic pain it has been shown that there is increased BDNF production in the dorsal horn of the spinal cord. Thus, it is possible that siRNA directed to BDNF delivered specifically to this site could hold promise as a pain therapeutic. However, other types of pain such as discomfort caused by any one of carpal tunnel syndrome pain, back pain, neck pain, sciatica, intercostal neuralgia, opioid resistant pain, trigeminal neuralgia, arthritis, osteoarthritis and cancer-related pain are encompassed by the invention.

Furthermore, it is envisioned that the methods of the invention could be used to affect gene expression following intermittent hypoxic episodes caused by for example sleep apnea, central hypoventilation syndrome, and apnea of prematurity. Episodes of intermittent systemic or local hypoxia affect metabolic pathways, initiate neuroplasticity, induce angiogenesis, and affect inflammatory responses. The inability of cells to detect and adapt rapidly to changes in oxygen may underlie various vascular, pulmonary, coronary, cerebral, and sleep disorder states.

Hypoxia has also been shown to modulate the activity of gene regulators, growth factors, and reactive oxygen species that serve as intermediary signals in the cellular response to oxygen level changes. In addition it is envisioned that the methods of the invention can be used for indirectly treating and preventing cyclic reductions in blood oxygen saturation during sleep apnea which is associated with a loss of upper airway patency and causes increased risk of hypertension, myocardial infarction, cerebrovascular condition, and neurocognitive deficits.

The following examples provide the materials and methods used to obtain and analyze the gene expression effects of the invention. These examples are intended to illustrate, but not limit, the present invention.

### Materials and Methods

### Experimental Animals

Experiments were conducted on adult male Sprague Dawley (Sasco colonies K62 and P04; Charles River Laboratories, Wilmington, Mass.) and Fisher rats (F344, colony 217; Harlan, Indianapolis, Ind.), weighing between 225-495 g. All experimental procedures were approved by the Institutional Animal Care and Use Committee at the University of Wisconsin-Madison.

In vivo Rat Preparation

Surgical procedures have been described elsewhere (7). Rats were anesthetized with isoflurane (2.5% in 50% O₂), vagotomized and pump-ventilated (Harvard Apparatus, South Natick, Mass.). The phrenic nerve was dissected, cut distally and desheathed. A laminectomy was performed over C₂, and a silicone catheter (2 French, Access Technologies; Skokie, Ill.) connected to a 50 μl glass syringe (Hamilton; Reno Nev.) was advanced through a small hole in the dura at C₂, such that the tip of the catheter lay over C₄-C₅. Following surgery, rats were converted to urethane anesthesia (1.6 g/kg, i.v.) and were paralyzed with pancuronium bromide (2.5 mg/kg). Supplemental doses of urethane were given as necessary. One hour after beginning surgery, an intravenous infusion of sodium bicarbonate solution (8.4%) and standard lactated Ringer's solution (1:4) was initiated to maintain acid-base balance (5 ml/kg/hr). End-tidal PCO₂ was monitored using a flow-through capnograph (Novametrix, Wallingford, Conn.). Periodically throughout the protocol, arterial blood was collected (0.3 ml) in a heparinized syringe to ensure that values remained constant (PaO₂<45 mmHg in hypoxia, PaO₂>120 mmHg in hyperoxia, PaCO₂±1 mmHg from baseline value). Arterial PCO₂ was corrected to the target range by adjusting ventilator frequency as necessary.

In one rat group, the carotid sinus nerve was transected bilaterally (CSNX) at the junction with the glossopharyngeal nerve (16). In a separate sham group, the carotid sinus nerves were identified, but not cut. Since CSNX rats did not receive drug treatments, laminectomy was not performed in these animals.

Respiratory-related activity was recorded in the phrenic nerve with a bipolar silver electrode. Nerve activity was amplified (A-M systems, Everett, Wash.), bandpass-filtered (100 Hz to 10 kHz), and integrated (CWE 821 filter; Paynter, Ardmore, Pa.). Phrenic activity was digitized, recorded and analyzed using the WINDAQ data acquisition system (DATAQ Instruments, Akron, Ohio).

Ninety minutes after surgery, stable integrated phrenic discharge was established in hyperoxia (baseline; 50% O₂, PaO₂>120 mmHg), with CO₂ added to the inspired gas so that PaCO₂ was 2-3 mmHg above the CO₂ apneic threshold (45 mmHg). In rats receiving intrathecal injections of vehicle or drug solutions, a bolus injection of 10-15 μl was given, and 30-35 min were allowed for drugs to penetrate the spinal cord prior to beginning protocols. Blood gases were measured before, during, and 15, 30 and 60 min post-intermittent hypoxia or hypercapnia, or at corresponding times in control rats (without hypoxia or hypercapnia).

### Measurements of Ventral Spinal BDNF Concentration

Inspired oxygen was manipulated to create intermittent hypoxia (the LTF protocol, which simulates repetitive apneas) or an equivalent duration of control oxygen levels. To compare with later studies using drug injections, a group of control and intermittent hypoxia exposed rats received intrathecal injections of vehicle (artificial CSF) 30 min prior to intermittent hypoxia (or equivalent time in control rats). Artificial CSF consisted of: 120 mM NaCl, 3 mM KCl, 2 mM CaCl, 2 mM MgCl, 23 mM NaHCO₃, 10 mM glucose aerated with 95% O₂/5% CO₂ to obtain appropriate pH (7.4). Control rats received 2 hrs isocapnic hyperoxia (50% O₂; n=9) to obtain baseline BDNF measurements. Rats receiving intermittent hypoxia were given 3, 5 min episodes of isocapnic hypoxia (11% O₂; n=12), separated by 5 min recovery periods (50% O₂). Following intermittent hypoxia, pre-hypoxia (baseline) conditions were restored (50% O₂; PaCO₂+1 mmHg from baseline) for 60 min. C₃-C₅ spinal segments were harvested at the end of the protocol and frozen for subsequent BDNF and NT-3 protein measurements. To confirm observed increases in ventral C₃-C₅ BDNF levels following intermittent hypoxia, BDNF was assayed in another rat group without intrathecal injections (controls, n=4; intermittent hypoxia, n=6).

BDNF protein concentration was measured in rat groups injected intrathecally with emetine (protein synthesis inhibitor; Sigma, St. Louis, Mo.) or methysergide maleate (serotonin receptor antagonist; Sandoz, Hanover, N.J.) 30 min prior to intermittent hypoxia or isocapnic hyperoxia. The C₃-C₅ spinal segments were harvested 60 min post-intermittent hypoxia (emetine, n=6; methysergide, n=8) or 2 hrs isocapnic hyperoxia (emetine control, n=3; methysergide control, n=9). Emetine or methysergide were dissolved in artificial CSF and delivered intrathecally at concentrations of 1 μg/kg (70 μM) or 250 μg/kg (20 mM), respectively (10-15 μl injected volume).

Ventral C₃-C₅ BDNF protein concentrations were determined 60 min post-intermittent hypoxia in rats with CSNX (n=11) or sham surgery (n=10). Baseline (control) BDNF measurements were made in corresponding groups exposed to 2 hrs isocapnic hyperoxia (CSNX, n=10; sham surgery, n=10).

To study the effects of intermittent hypercapnia, stable phrenic discharge (see above, 50% O₂; arterial PCO₂˜45 mmHg) was established and the inspired CO₂ was manipulated to create three 5-min episodes of hypercapnia (arterial PCO₂˜85 mmHg), separated by 5-min recovery periods (n=6). Following intermittent hypercapnia, rats were returned to baseline conditions (arterial PCO₂±1 mmHg from baseline). Throughout the protocol, inspired oxygen was maintained at 50% O₂. Ventral C₃-C₅ BDNF levels were analyzed in tissues harvested 60 min after intermittent hypercapnia or after 75 min of normocapnia (controls; n=3).

En bloc spinal cord segments C₃-C₅ were placed on a freezing microtome, and successive 50 μm sections of the dorsal horn were removed and discarded until the ventral aspect of the central canal was visible. BDNF and/or NT-3 analyses were performed on the remaining ventral spinal cord. Tissue samples were weighed and homogenized in cold extraction buffer (Tris-buffered saline, pH 8.0, with 1% NP-40, 10% glycerol, 5 mM sodium metavanadate, 10 mM PMSF, 100 μg/ml aprotinin and 10 μg/ml leupeptin). Homogenates were acidified with 1 N HCl (pH ˜3.0), incubated at room temperature for 15 min, and neutralized with 1 N NaOH (pH ˜7.6). Homogenates were then microfuged at 7000 g for 10 min, and the supernatants were assayed with antibody sandwich ELISAs (BDNF ELISA, R & D Systems, Minneapolis, Minn.; NT-3 ELISA, Promega Corporation, Madison, Wis.). Neurotrophin concentrations were normalized per gram of tissue wet weight and per gram of total protein determined with the BCA (bicinchoninic acid) method (Pierce, Rockford, Ill.). Since both normalization methods produced qualitatively similar results neurotrophin concentrations are presented only per gram of tissue wet weight.

### Phrenic Activity Following Intrathecal BDNF Injections

An intrathecal catheter was placed and 10-13 μl solutions of BDNF (0.1 μg, n=5) or vehicle (artificial CSF+0.1% BSA; n=5) were injected. To determine if intrathecal BDNF injections unintentionally spread beyond the spinal cord to cranial motor pools, the hypoglossal nerve was also dissected, desheathed and recorded. Hypoglossal and phrenic activity were observed for 90 min post-injection. In another group of rats, two intrathecal catheters were placed over C₄, and a bolus injection of the tyrosine kinase inhibitor K252a was given 10 min prior to BDNF injection (BDNF+K252a; BDNF=0.1 μg; K252a=0.15 microgram, n=3). Recombinant human BDNF (Promega Corporation, Madison, Wis.) was dissolved in artificial CSF+0.1% bovine serum albumin (BSA). K252a (Calbiochem; San Diego, Calif.) was initially dissolved in dimethylsulfoxide (DMSO; 100 μg K252a in 1.5 ml DMSO) and frozen in 200 μl aliquots. Prior to experimentation, 0.8 ml artificial CSF was added to a 200 μl aliquot of K252a/DMSO solution, of which 10-15 μl was delivered intrathecally (final concentration of K252a=0.13-0.2 μg; 28 μM). Vehicle delivery was as described above (DMSO+artificial CSF), except K252a was not added. At 30, 60 and 90 min post-injection, arterial blood gases were analyzed to ensure that PaO₂ and PaCO₂ levels were similar to pre-injection values.

siRNA Design and Synthesis

In order to down regulate hypoxia induced BDNF synthesis in vivo, as described herein, small interfering RNAs (siRNA) directed against BDNF mRNA or a scrambled sequence were designed and synthesized by Dharmacon, Inc. (Lafayette, Colo.). The BDNF siRNA consisted of 4 pooled 21-nucleotide duplexes with symmetrical 3′ overhangs (SMARTpool). The sequences of the 4 duplexes were as follows: 1) TCGAAGAGCTGCTGGATGA (SEQ ID NO: 1); 2) TATGTACACTGACCATTAA (SEQ ID NO: 2); 3) GAGCGTGTGTGACAGTATT (SEQ ID NO: 3); and 4) GAACTACCCAATCGTATGT (SEQ ID NO: 4). BDNF siRNA and scrambled siRNA were suspended in siRNA Universal Buffer (Dharmacon; Layfayette, Colo.) to yield a concentration of 50 μM. The siRNA stocks were aliquotted and stored at 20° C. As an alternative approach to regulating BDNF-induced hypoxia, it is also envisioned that siRNAs directed against the mRNA of individual protein phosphatases (PP1, PP2A, PP2B, and PP2C) could be developed, as described herein, for in vitro or in vivo applications or kinases (e.g., ERK1/2, CAMKII, PKC, PKA, MEKK) or translation factors.

It is further envisioned that the siRNAs of the invention may be chemically or enzymatically synthesized, as described in WO 99/32619 and WO 01/68836. Enzymatic synthesis of siRNA may use a cellular RNA polymerase or a bacteriophage RNA polymerase (e.g. T3, T7, SP6) facilitated by expression constructs known in the art, such as for example described in U.S. Pat. No. 5,795,715. The contemplated constructs provide templates that produce RNAs which contain nucleotide sequences identical to and complementary to a portion of the target gene representing the sense and antisense strands, respectively. The length of identical sequences provided by these references is at least 25 base pairs in length. This method contemplates digesting longer dsRNAs to 19-25mer lengths with the endogenous nuclease complex that converts long dsRNAs to siRNAs in vivo. No distinction is made between the expected properties of chemical or enzymatically synthesized dsRNA for its use in RNA interference.

Applicants place no limitation upon the manner in which the siRNA is synthesized, providing that the RNA may be synthesized in vitro or in vivo, using manual and/or automated procedures. The references described hereinabove also provide that in vitro synthesis may be chemical or enzymatic, for example using cloned RNA polymerase (e.g. T3, T7, SP6) for transcription of the endogenous DNA (or cDNA) template, or a mixture of both. Again, no distinction in the desirable properties for use in RNA interference is made between chemically or enzymatically synthesized siRNA.

siRNA in vitro

Murine HT-22 hippocampal cell line (Salk Institute, San Diego, Calif.) was grown to 80% confluency and passaged in Dulbecco's modified Eagle's medium supplemented with 10% fetal bovine serum and 100 units/ml penicillin/streptomycin. Cells were plated overnight in Falcon 6 well plates at a density of 2×10⁵ cells/well. The following day, cells were transfected with 200 nM scrambled or BDNF siRNA. Twenty minutes prior to transfection, siRNAs (BDNF or scrambled) or siRNA Universal Buffer was combined with Oligofectamine™ (0.6 microliter from stock; Invitrogen, Carlsbad, Calif.) and added to the appropriate wells. The effects of the individual BDNF siRNA duplexes on BDNF mRNA were studied in a similar manner. Each treatment (buffer, scrambled siRNA and BDNF siRNA) was performed in duplicate.

Applicants also encompass that these in vitro experiments may be conducted with BDNF siRNA having siSTABLE™ modification (available through Dharmacon, Lafayette, Colo.). siSTABLE™ is a proprietary form of siRNA with chemically modified strands that enhance stability and silencing longevity without compromising efficacy or increasing cellular toxicity.

Twenty-four hours following treatment, total RNA was extracted from HT-22 cells. cDNA was synthesized using M-MLV Reverse Transcriptase (Invitrogen; Carlsbad, Calif.). 18s rRNA was used to normalize BDNF mRNA values. Gene sequences were obtained from Genbank using the Unigene search engine (maintained by the National Center for Biotechnology Information). Primer sequences were designed using Primer Express software version 2.0 (Applied Biosystems, Inc., Foster City, Calif.) and synthesized by Integrated DNA Technologies (Coralville, Iowa). BDNF forward primer: 5′-CTGACACTTTTGAGCACGTGATC-3′ (SEQ ID NO: 5); reverse primer: 5′-AGGCTCCAAAGGCACTTGACT-3′ (SEQ ID NO: 6); 18s Ribosomal Subunit forward primer: 5′-AACGAGACTCTCGGCATGCTAA-3′ (SEQ ID NO: 7); reverse primer: 5′-CCGGACATCTAAGGGCATCA-3′ (SEQ ID NO: 8). The relevant Genbank Accession numbers are as follows: BDNF, NM_{—}012513; and 18s Ribosomal Subunit, X01117 K01593.

Quantitative RT-PCR was performed using an Applied Biosystems Model ABI 7000 Prism Sequence Detection System (Applied Biosystems, Inc.). An RT-PCR reaction volume of 25 μl was used. All samples were run in duplicate using an annealing temperature of 60° C. Data were collected and analyzed using the Comparative CT Method. Primer specificity was confirmed by dissociation (melting) curve analysis and agarose gel electrophoresis.

SiRNA in vivo

Control rats or rats treated with scrambled sequence or BDNF siRNA were surgically prepared as described above. The siRNA (17 μl) was combined with Oligofectamine™ (2.5 μl) to make a siRNA composition. The siRNA composition was incubated at room temperature (22-24° C.) for 15 min. Rats were anesthetized with isoflurane and the diaphragm exposed through a small ventral midline incision. The rats received 10, 4 μl bilateral diaphragm injections of siSTABLE™ siRNA (BDNF or scrambled) or vehicle. The siRNA composition was injected over C₄ immediately following spinal cord exposure (two ˜10 microliters injections spaced one minute apart). Two hours were allowed prior to initiating the protocol (described above), and tissues were collected 60 min following the final hypoxic episode (control n=3, scrambled siRNA n=3, BDNF siRNA n=4). For baseline BDNF measurements, tissues were collected 3.5 hours following siRNA injections without receiving hypoxia (control n=3, scrambled siRNA n=3, BDNF siRNA n=4). Ventral gray matter from C₄-C₅ segments was isolated by removing the dorsal half of the spinal cord using a freezing microtome, then isolating the ventral gray matter using a small dissection knife. BDNF protein concentration in ventral C₄-C₅ gray matter was determined via ELISA. The effect of siRNA on pLTF was determined in another rat group receiving scrambled siRNA (n=3) or the BDNF siRNA pool (n=5) two hours prior to intermittent hypoxia. In four additional rats, individual siRNA duplexes were administered as above (n=2 each; duplexes 1 and 4) and pLTF was measured.

### Intermittent and Sustained Hypoxia.

Air, O₂ and/or N₂ were mixed to achieve the desired pattern of inspired oxygen concentration. In anesthetized rats, intermittent hypoxia consisted of three 5 min episodes of 11% O₂, separated by 5 min intervals, since this pattern has been shown to elicit LTF and lead to increased BDNF protein levels 60 min post-hypoxia. Sustained hypoxia consisted of 25 min of 11% O₂, a protocol which does not elicit LTF (Baker and Mitchell, 2000). Control groups receiving similar surgery or treatments, but not receiving hypoxia, exposed parallel rats to hypoxia.

Awake rats were exposed to hypoxia in an environmental chamber designed in-house. The chambers were approximately 4 L in volume. Computer mixed gases were passed through the chamber at a flow rate of 4 L/min per chamber to assure levels of C O₂ accumulation below 0.5%, and to enable rapid dynamics in the on and off transients during hypoxic episodes (50 and 70 sec for down and up transients, respectively). Intermittent hypoxia consisted of 5, 5 min episodes of 9-10% O₂, separated by 5 min intervals, since this pattern has been shown to elicit LTF in awake rats. Sustained hypoxia consisted of 25 min of 9-10% O₂.

pLTF Following Intrathecal K-252a

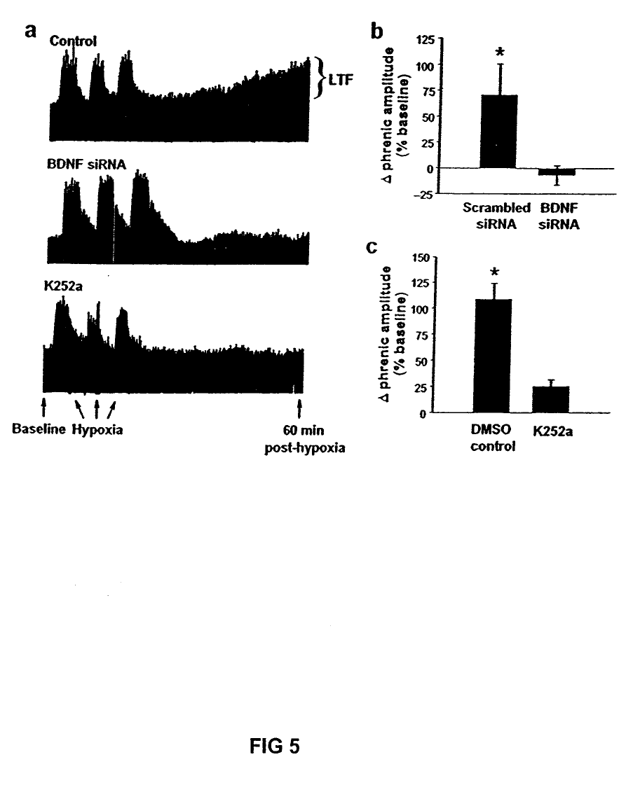

Intrathecal injections of K252a were performed prior to intermittent hypoxia; K252a was prepared as described above. Intrathecal K252a (n=6) or vehicle (DMSO+artificial CSF; n=5) was delivered 30 min prior to intermittent hypoxia, and phrenic discharge was monitored continuously for 60 min post-intermittent hypoxia. Phrenic activity was expressed as a percentage of baseline (pre-hypoxia) values (see FIG. 5).

Peak integrated phrenic amplitude was averaged in 30-second bins before (baseline) and 60 min post-hypoxia or hypercapnia, and pLTF magnitude was calculated at 60 min post-hypoxia or hypercapnia as a percentage change of the baseline value. Regression analysis was performed to determine the relationship between pLTF magnitude and the percentage change in BDNF from controls. For this analysis, pLTF was averaged and the change in BDNF from controls within each experimental condition is presented herein.

A one-sample t-test was used to determine if rats pretreated with DMSO, K252a, scrambled siRNA or BDNF siRNA had significant pLTF. A Student's t-test was used to compare pLTF in the treatment groups with the controls (DMSO versus K252a, scrambled versus BDNF siRNA). A two-way ANOVA with a repeated measures design was used to compare phrenic responses before and after BDNF, vehicle and BDNF+K252a injections, and individual comparisons were made with the Student-Neuman-Kuels post hoc test. Differences were considered significant if P<0.05. All values are expressed as mean±standard error.

Delivery of siRNA Molecules into Tongue Muscle

In this embodiment of the invention, an siRNA composition as described above directed to the BDNF target gene was injected into a rat tongue. The rat, Fisher (F344) was obtained from Harlan Sprague-Dawley, (Indianapolis, Ind.). The siRNA molecules were obtained from Dharmacon and consisted of four, 21-nucleotide duplexes as described herein. A stock of siRNA (50 μl) was combined with 7.5 μl Oligofectamine™ (Invitrogen; Carlsbad, Calif.) forming an siRNA composition. The siRNA composition was maintained for 15 min at room temperature prior to injection into the rat tongue. Carprofen was administered subcutaneously (5 mg/kg) upon anesthesia induction to reduce pain. A series of eight, 7 μl injections of the BDNF siRNA compositions was made in the tongue, covering the top, bottom and base of the tongue bilaterally. The rat was allowed to recover for 2 days prior to being analyzed. The rat was then subjected to the LTF protocol as described above. The LTF was shown to be blocked 2 days later.

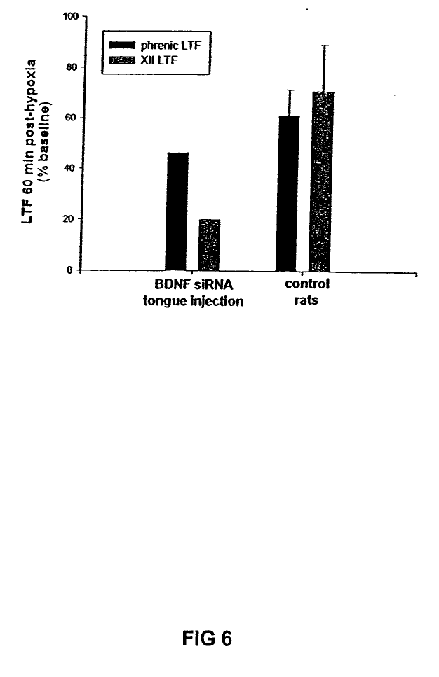

The results of the analysis are provided in FIG. 6, which shows a graph of the functional result following tongue injection of BDNF siRNA molecules. The results suggest that the form of BDNF-dependent plasticity (LTF) was blocked in the targeted motoneuron pool, in the XII nucleus, but not blocked in a related (but separated) phrenic motor output. This indicates that the siRNAs were likely confined to the motoneuron pool that innervates the tongue XII nucleus and did not reach other, related targets.

This experiment demonstrated that the BDNF siRNA can be transported to the motoneuron cell body from the tongue muscle, blocking BDNF functions. This approach allows delivery of siRNA to an accessible, peripheral site, the muscle, but affects nerve cells (motoneurons) located in the nervous system behind the blood brain barrier. It is envisioned that this approach will be applicable to different molecular targets of siRNA, allowing alterations of gene expression in a well defined cell, suitably motoneuron, which play s a role in neuro-muscular disorders such as sleep apnea, spinal cord injury, ALS and polio.

Delivery of siRNA into Diaphragm Muscle

It is also envisioned that similar to the above-described tongue injections, siRNA compositions may be injected into the diaphragm muscle of a mammal, as well as in virtually any muscle in the body. In this embodiment of the invention, an siRNA composition directed to the BDNF target gene is injected into a rat diaphragm muscle to reach phrenic motoneurons. Prior to the diaphragm injections, the siRNA compositions will be prepared by combining an siRNA stock (50 μl) with 7.5 microliters Oligofectamine™ (RNase free conditions) and maintaining it at room temperature for 15 minutes. Carprofen was administered subcutaneously (5 mg/kg) immediately upon anesthesia induction. A midline incision was made to expose the diaphragm. Then, a series of twelve, approximately 5 microliters injections of siRNA compositions (i.e., BDNF siRNA, scrambled siRNA or saline) was made in the diaphragm, covering both hemidiaphragms. Rats were recovered for 2-5 days prior to the LTF protocol. The BDNF siRNA compositions injected into the rat diaphragm reached the phrenic motoneurons within three days, and affected spinal respiratory plasticity following intermittent hypoxia.

To measure phosphatase activity, tissue samples were homogenized in 1 ml phosphatase storage buffer (50 mM Tris-HCl, pH 7.5, 1 mM EDTA, 0.1% β-mercaptoethanol, 100 mM leupeptin, 75 mM pepstatin) and centrifuged for 1 hr at 100,000×g at 4° C. Phosphatase activity was immediately quantified in the supernatants as described hereinbelow.

For mRNA analyses, the XII nucleus and ventral gray matter were homogenized in 250 ml RNA extraction reagent (Trizol Reagent, Invitrogen), respectively, according to manufacturers instruction. In brief, chloroform (⅕ Trizol volume) will be added to the samples to denature proteins. After a 3 min incubation at room temperature, samples will be centrifuged (12,000 g, 4° C., 15 min) to separate organic and inorganic layers. The organic layer will be collected and isopropanol (½ Trizol volume) will be added to precipitate RNA (10 min room temperature incubation). The RNA will be spun to a pellet (12,000 g, 4° C., 10 min), washed with 500 ml of ice-cold 80% ethanol and centrifuged twice (12,000 g, 4° C., 5 min) to remove excess ethanol. Pellets will be air-dried for 10 min and re-suspended in 30-50 ml DEPC-treated water. RNA samples will be stored at −80° C. for later analysis.

### ELISA

A BDNF ELISA (R&D Systems) was used to quantify BDNF changes following treatments. In brief, tissue samples and a standard curve will be added to a 96-well polystyrene plate pre-coated with a BDNF monoclonal antibody. The plate will then be incubated for 2 hrs at room temperature to allow BDNF in the experimental and standard samples to bind to the immobilized antibody. An enzyme-linked (horseradish peroxidase) BDNF monoclonal antibody will be added and incubated for 1 hr at room temperature, during which the conjugated antibody forms a “sandwich” with the immobilized antibody-BDNF protein complex. The plate will then be washed with buffer, and a substrate solution (hydrogen peroxide+chromogen) added. Color is allowed to develop for 30 min, and then an acidic solution (2 N sulfuric acid) is added to stop the peroxidase reaction. The color intensity is measured using a microplate reader (MRXI Absorbance Reader with MRX Revelation software; Dynex Technologies) set to 450 nm with wavelength correction at 570 nm. The absorbances are directly proportional to the amount of bound BDNF. BDNF protein levels in the samples are normalized to both total protein levels and per gram of tissue, wet weight. Total protein is determined using a BCA assay kit (Pierce Biotechnology).

### Immunoblot Analyses

Samples were diluted 1:2 with 2× sample buffer (20 mM Tris, 2 mM EDTA, 1 mM Na₃VO4, 2 mM dithiothreitol, 2% SDS, 20% glycerol) and boiled (105° C.) for 5 minutes. Equal amounts of protein (˜30 mg) from each sample were loaded per lane and separated by 10% SDS-PAGE gel. Proteins in the gels were transferred to Immobilon polyvinylidene difluoride (PVDF) membrane (Millipore Corp.). Membranes were blocked in 5% non-fat milk/TBST (10 mM Tris-HCl, pH 8, 150 mM NaCl, 0.05% Tween 20) for 30 min at 37° C. An anti-phosphorylated ERK1/2 MAP kinase antibody (Cell Signaling Technology), which recognize enzymatically active ERK1/2, was used at a dilution of 1:2500 overnight at 37° C. The immunoreactive bands were visualized using secondary antibodies conjugated to horseradish peroxidase (goat anti-rabbit IgG-HRP; Santa Cruz Biotechnology) at a dilution of 1:8000 and chemiluminescent detection with SuperSignal West Pico Chemiluminescent Substrate (Pierce Biotechnology). Band density will be determined using the AC1 AutoChemi System (UVP) and quantified using LabWorks Image Acquisition and Analysis Software (UVP). All samples were run in duplicate. Following imaging of phosphorylated ERK1/2 MAP kinase immunoreactive bands, membranes were stripped using Restore Western Blot Stripping Buffer (30 min at 37° C.; Pierce Biotechnology), re-blocked in 5% milk/TBST, and probed using an anti-ERK1/2 MAP kinase antibody that recognizes both phosphorylated and non-phosphorylated forms of ERK1/2 (1:2500 dilution; Santa Cruz Biotechnology). The density of phosphorylated ERK1/2 MAP kinase was normalized to total ERK1/2 MAP kinase within the same lane; the percentage change in normalized phosphorylated ERK1/2 MAP kinase in treated versus control rats run on the same gel was then calculated.

### Protein Phosphatase Activity

Protein phosphatase activity was assayed using the Serine/Threonine Phosphatase Assay System (Promega Corporation) according to manufacturers instructions. In brief, free phosphates in the samples will be removed using a Sephadex G-25 resin column and centrifuged at 600 g at 4° C. for 5 min. A reaction buffer that preferentially targets protein phosphatase 2A (PPTase-2A 5× reaction buffer: 10 ml; 250 mM imidazole, pH 7.2, 1 mM EGTA, 0.1% β-mercaptoethanol, 0.5 mg/ml BSA) and 1 mM phosphopeptide (5 ml) will be added to a 96 well plate and incubated at 30° C. for 3 min. The sample lysate (5 ml) diluted in phosphatase storage buffer (30 ml; see tissue preparation section) was added to the wells and incubated for 30 min at 30° C. Enzymatic activity of the protein phosphatases in the sample lysate was stopped with Molybdate Dye/Additive mixture (50 ml), and the plate will be incubated at room temperature for 15 min. Optical density of the samples was read at 630 nm using a microplate reader (MRXI Absorbance Reader with MRX Revelation software; Dynex Technologies). The level of serine/threonine phosphatase activity in each sample was calculated using a standard curve generated by diluting the phosphate standard. To compare across samples, the level of phosphatase activity was divided by total protein in the sample lysates (determined using the Pierce BCA protein assay kit).

### Statistical Comparisons and Analysis

For BDNF measurements, each treatment group was paired with age-matched control rats with similar surgery and drug treatments, but did not receive intermittent hypoxia or hypercapnia. To analyze changes in BDNF, a Student's t-test was used to detect significant differences between matched controls and experimental groups exposed to intermittent hypoxia or hypercapnia. To compare among groups, BDNF levels following intermittent hypoxia or hypercapnia were normalized as a percentage change from the appropriate controls prepared and analyzed on the same day (to control for batch differences). For example, BDNF concentration following hypoxia in rats pretreated with methysergide were expressed as percentage changes from the average BDNF concentration in methysergide controls homogenized with the same lysis buffer and analyzed on the same ELISA plate. A one-way ANOVA was used to test for statistical differences in the percent change in BDNF across experimental groups. A two-way ANOVA was used to make statistical inferences regarding baseline BDNF concentrations in control (no drug+scrambled siRNA) and BDNF siRNA treated rats.

### EXAMPLES

### Example 1

### Intermittent Hypoxia Regulates Ventral Cervical BDNF

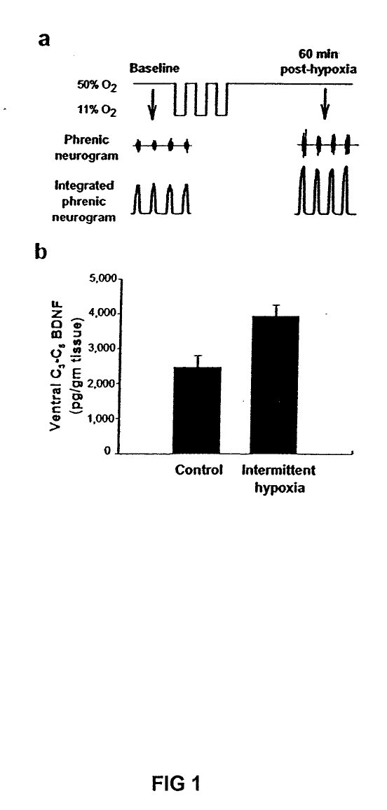

In accordance with the invention it was determined that intermittent hypoxia elicits pLTF and increased BDNF synthesis in ventral spinal segments associated with the phrenic motor nucleus. This was identified initially through the observation that sixty minutes following intermittent hypoxia in anesthetized rats, peak integrated phrenic activity increased above pre-hypoxia (baseline) levels indicating pLTF (3, 4, 6) (FIG. 1A). To determine if changes in BDNF or NT-3 protein concentration were associated with pLTF, BDNF and NT-3 levels were analyzed in ventral cervical segments encompassing the phrenic motor nucleus (C₃-C₅). In rats administered intrathecal artificial CSF, BDNF protein concentration in ventral C₃-C₅ increased from 2476±321 pg/g tissue in control rats to 3940±308 pg/g tissue 60 min post-intermittent hypoxia (P<0.05; FIG. 1B). Thus, intermittent hypoxia increased BDNF protein concentration near the phrenic motor nucleus 56±12% (batch controlled; P<0.05). This observation was confirmed in rats exposed to intermittent hypoxia, but without intrathecal aCSF; BDNF concentration increased 49±9% 60 min post-intermittent hypoxia (P<0.05; data not shown). In contrast, ventral cervical NT-3 concentration was unaffected by intermittent hypoxia (control: 2397±457; 60 min post-hypoxia: 2291±389 pg/g tissue; P>0.05). It is envisioned that the intermittent hypoxia also increases BDNF synthesis in other respiratory motor nuclei (for example, hypoglossal) as well as non-motor nuclei involved in other functions such as walking, posture, reaching and grasping or speech.

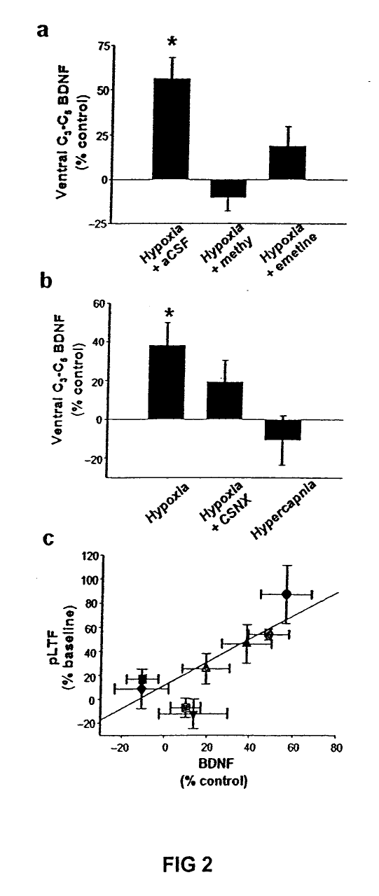

To determine if increased BDNF following intermittent hypoxia was regulated by mechanisms similar to pLTF (7), intrathecal injections of a serotonin receptor antagonist (methysergide) or protein synthesis inhibitor (emetine) were administered in the cervical spinal cord prior to intermittent hypoxia. Intrathecal methysergide (250 microgram/kg) blocked intermittent hypoxic effects on ventral cervical BDNF concentration (change from methysergide control, −10±8%, P>0.05; FIG. 2A). Increased BDNF following intermittent hypoxia also required new protein synthesis since pretreatment with emetine (1 μg/kg) attenuated BDNF increases 60 min post-hypoxia (change from emetine control, 19±11%, P>0.05; FIG. 2A). It is envisioned that similar mechanisms may regulate BDNF in other respiratory and non-respiratory motor nuclei.

As shown in FIG. 2, rats with intrathecal artificial CSF injections had a significant increase in ventral C₃-C₅ BDNF concentration 60 min following intermittent hypoxia. Intrathecal methysergide or emetine blocked BDNF increases 60 min post-intermittent hypoxia, suggesting a serotonin-dependent increase in BDNF synthesis. Thus, intermittent hypoxia regulates ventral cervical BDNF concentration by a serotonin-dependent increase in BDNF synthesis.

The primary oxygen-sensitive chemoreceptors in adult mammals are in the carotid body. In carotid denervated rats (CSNX), intermittent hypoxia elicited a non-significant 19±11% increase in ventral C₃-C₅ BDNF concentration (P>0.05; FIG. 2B). Although sham CSNX rats increased ventral cervical BDNF 38±12% post-intermittent hypoxia (P<0.05; FIG. 2B), this value was not significantly different from changes in CSNX rats (P>0.05). Nevertheless, intact chemoreceptors appear necessary for the full effect of intermittent hypoxia on ventral cervical BDNF.

Ventral C₃-C₅ BDNF concentration was measured following intermittent hypercapnia to determine if increased BDNF concentration is a nonspecific response to increased respiratory (synaptic) activity. Although hypercapnia is a powerful respiratory stimulus, it does not elicit pLTF (3,13). The BDNF concentration was unchanged, 60 minutes post-intermittent hypercapnia, (change from control −10±13%, P>0.05; FIG. 2B). In referring to FIG. 2B, it suggests that increased BDNF in ventral C₃-C₅ following intermittent hypoxia at least partially requires carotid chemoreceptors, since intermittent hypoxia failed to significantly increase BDNF following CSNX. Increased BDNF was not observed 60 min following intermittent hypercapnia, indicating that the effect is not a general response to increased respiratory drive. Thus, it was found that intermittent hypoxia exerts a unique influence on ventral spinal BDNF synthesis.

It is noted that regression analysis on mean data from studies described herein revealed a strong correlation (R²=0.67, P=0.01; FIG. 2C) between the percentage change in BDNF (from controls) and the magnitude of pLTF. Rats with the largest BDNF increase had the largest pLTF.

### Example 2

### Intrathecal BDNF Elicits Long-Lasting Phrenic Facilitation

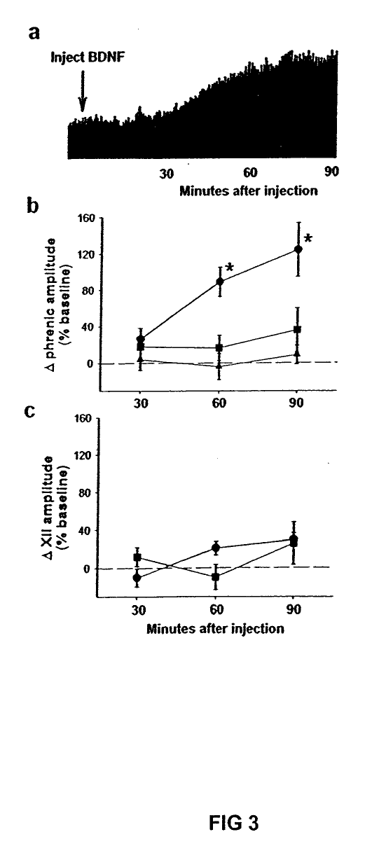

Applicants also determined that intrathecal BDNF is sufficient to facilitate phrenic motor-output and elicit pLTF. In order to make this determination, rats were injected with BDNF (0.1 μg) in the intrathecal space above the phrenic motor nucleus. Whereas vehicle (artificial CSF+0.1% BSA) injections elicited no time-dependent change in phrenic activity (90 min post-injection: 36±24% above baseline, P>0.05; FIG. 3B), intrathecal BDNF significantly increased integrated phrenic discharge (125±25%, 90 min post-injection, P<0.05; FIG. 3A,B).

Intrathecal BDNF elicited significant increases in phrenic burst amplitude 60 and 90 min following injection, and this effect was blocked by K252a. BDNF-induced facilitation was blocked by pre-treatment with intrathecal K252a (9±10%, 90 min post-injection, P>0.05; FIG. 3B), a Trk receptor inhibitor. Intrathecal BDNF effects were restricted to the spinal cord since there were no time-dependent changes in hypoglossal nerve activity, a reflection of brainstem respiratory motor output (30±17%, 90 min post-injection, P>0.05; FIG. 3C). Thus, spinal BDNF facilitates phrenic motor output, likely via TrkB receptor activation.

### Example 3

### BDNF siRNA Blocks pLTF

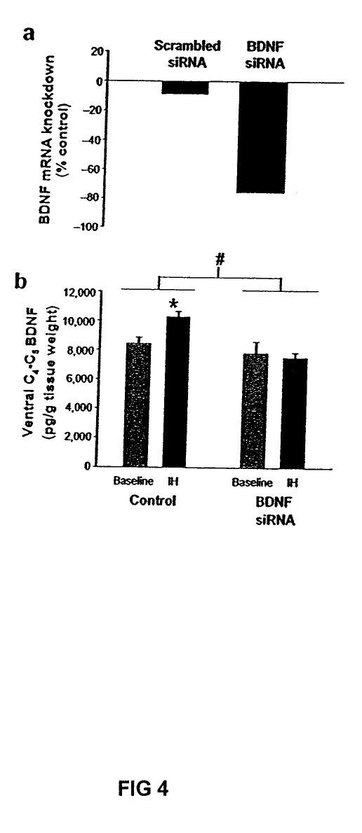

Furthermore, applicants found that BDNF siRNA reduced BDNF mRNA in vitro and hypoxia-induced BDNF synthesis in vivo as described in the examples above. In making this determination the role of endogenous BDNF in pLTF was tested using RNA interference (RNAi). RNA interference is achieved with double-stranded RNA segments that elicit sequence specific inhibition or degradation of homologous mRNA via an endogenous pathway (14,15). Small, synthetic segments of double-stranded RNA (small interfering RNA; siRNA) directed against BDNF mRNA were used to reduce BDNF mRNA translation induced by intermittent hypoxia. Rather than degrading BDNF mRNA per se, the goal was to impair BDNF mRNA translation sufficiently to minimize new BDNF synthesis following intermittent hypoxia. To assure effectiveness of the siRNA sequences used, the ability of these siRNAs to knock-down BDNF mRNA was determined in vitro. In a hippocampal cell line (HT-22), BDNF siRNA (200 nM) reduced BDNF mRNA 75% 24 hours post-transfection, an effect not seen with scrambled duplexes (FIG. 4A). The pooled duplexes were also tested individually, each individual duplex in the BDNF siRNA pool decreased BDNF mRNA in HT-22 cells 24 hours post-transfection. Three of the four tested duplexes knocked down BDNF mRNA by more than 50%.

The effect of BDNF siRNA inhibition of BDNF synthesis following intermittent hypoxia was then investigated in anesthetized rats. There were no significant differences in BDNF protein concentration in the C₄-C₅ ventral gray matter in rats without injection versus scrambled siRNA injection during baseline conditions (8293±479 vs. 8588±783 pg/g tissue wet weight, respectively). Likewise, there was no significant difference between uninjected rats and rats injected with scrambled SiRNA 60-min post-intermittent hypoxia (10307±490 vs. 10282±592 pg/g tissue wet weight, respectively, P>0.8). Both groups were combined for analysis and designated as control rats. There was no significant difference in baseline (no hypoxia) BDNF protein concentration in control rats versus rats receiving BDNF siRNA (8440±416 vs. 7818±761 pg/g tissue wet weight, respectively, P=0.4; FIG. 4B), suggesting that BDNF siRNA did not knock-down basal levels of BDNF protein within this short time frame (3 hours). In contrast, while control rats had significantly increased BDNF protein concentrations in C₄-C₅ ventral gray matter 60 min post-intermittent hypoxia (baseline: 8440±416 pg/g tissue wet weight, 60 min post-hypoxia 10295±344 pg/g tissue wet weight, P<0.05), rats treated with BDNF siRNA did not (baseline: 7818±761 pg/g tissue wet weight, 60 min post-hypoxia 7529±318 pg/g tissue wet weight, P>0.05; FIG. 4B).

As shown in FIG. 4B, BDNF siRNA did not knock-down basal BDNF protein levels in this short time frame (gray bars). However, BDNF siRNA prevented hypoxia-induced increases in BDNF protein concentration (black bars).

To determine the effect of BDNF siRNAs (and lack of increased BDNF synthesis) on pLTF following intermittent hypoxia, rats received intrathecal injections of either a scrambled siRNA sequence or the BDNF siRNA pool (10 mg) while phrenic activity was recorded. Rats receiving scrambled siRNA injections expressed significant pLTF 60 min post-intermittent hypoxia (71±30% above baseline, P<0.05; FIG. 5B). In contrast, rats receiving the BDNF siRNA pool did not express pLTF (−7±9%, P<0.05; FIG. 5A,B).

In referring to FIG. 5A, a typical rat exhibited a progressive increase in phrenic amplitude (pLTF) for at least one hour following intermittent hypoxia (upper trace). In contrast, a rat pretreated with intrathecal BDNF siRNA or K252a showed no pLTF (bottom two traces).

In these same rats, there was no detectable increase in ventral C₃-C₅ BDNF protein concentration 60 min post-intermittent hypoxia (10±7% change from rats with BDNF siRNA but without hypoxia, P>0.05). To ensure specificity of the BDNF siRNA pool in vivo, two individual BDNF siRNA duplexes were tested for their effect on pLTF. One hour post-intermittent hypoxia, pLTF averaged 23% and 27% for duplexes 1 and 4 (see Methods for duplex sequences), respectively. Both values are below the lower limit of the 95% confidence interval for pLTF in control rats (67% to 125%). Collectively, these data demonstrate that BDNF siRNAs inhibit hypoxia-induced BDNF mRNA translation and pLTF, providing compelling evidence that endogenous BDNF synthesis is necessary for pLTF following intermittent hypoxia. It is envisioned that BDNF is necessary for LTF or motor plasticity in other motor nuclei as well.

### Example 4

### pLTF Requires Trk Receptor Activation

Additionally, it was found that BDNF siRNA and Trk receptor inhibition with K252a block pLTF. This finding was determined by testing the hypothesis that pLTF requires activation of a high affinity Trk receptor. Rats were pretreated with intrathecal K252a (0.13-0.2 μg), a non-specific Trk receptor inhibitor. In rats receiving intrathecal DMSO (vehicle), integrated phrenic burst amplitude was significantly increased from baseline 60-min post-intermittent hypoxia (109±19% above baseline, P<0.05; FIG. 5A,C), indicating pLTF. In contrast, rats pretreated with K252a had no significant increase in phrenic burst amplitude 60 min post-intermittent hypoxia (25±6%, P>0.05; FIG. 5A,C), a response significantly lower than in rats injected with DMSO alone (P<0.05). Thus, Trk receptor activation is necessary for full expression of pLTF following intermittent hypoxia, which is consistent with the hypothesis that BDNF acts via the TrkB receptor to elicit pLTF.

### Example 5

### Diaphragm Injection of BDNF siRNA Blocks Phrenic LTF

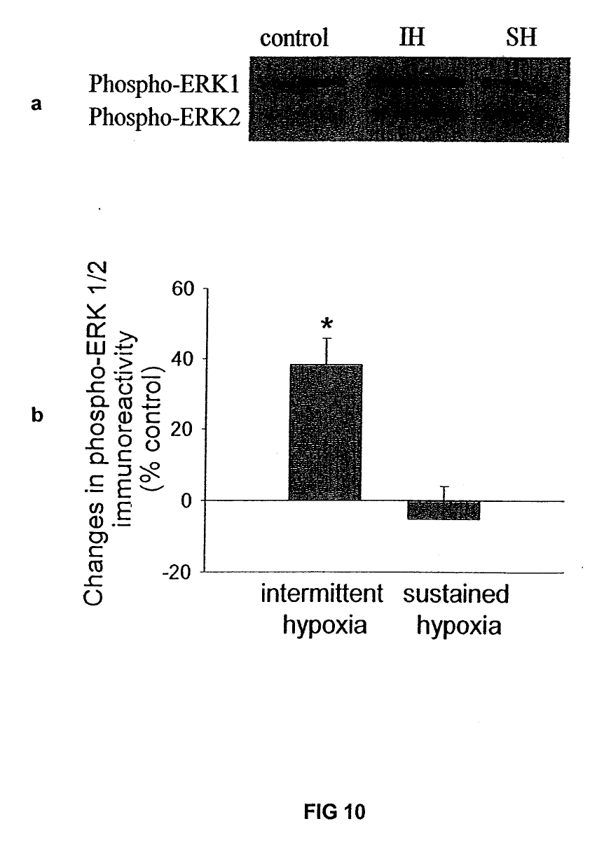

In accordance with the invention, experiments were performed to confirm the hypothesis that BDNF synthesis within phrenic motoneurons is necessary for pLTF and to show the feasibility of retrograde transport of siRNAs from a target muscle to the motoneurons that innervate that muscle. To do this, BDNF siRNAs were targeted to the phrenic motoneurons by siRNA injections into the diaphragm to prevent hypoxia-induced BDNF synthesis within phrenic motoneurons per se. In preliminary experiments, applicants delivered BDNF siRNAs to the diaphragm for axonal uptake and retrograde transport. FIG. 10 depicts exciting preliminary data from one rat that received diaphragm injections of BDNF siRNA (10, 4 ml injections; 50 ml of a 50 mM solution of two siRNA duplexes added to 8 ml Oligofectamine™). Three days post-injection, pLTF was greatly attenuated, but LTF was still observed in a distant respiratory motor pool (XII). In a parallel experiment, BDNF siRNA was injected into the tongue, and applicants observed the reverse result (i.e. XII LTF was abolished, but pLTF was not; FIG. 6). Collectively, these data strongly suggest that, following intramuscular injection, BDNF siRNAs are transported to the target motoneurons where they degrade/inhibit BDNF mRNA and prevent LTF. Successful delivery of the siRNA should reduce BDNF protein levels due to normal protein turnover, and reduce hypoxia-induced translation of BDNF mRNA. Applicants envision that this technique could be further developed by characterizing BDNF mRNA and protein changes following diaphragm injections of BDNF siRNA, as well as further experiments concerning the functional consequences on pLTF. Applicants also envision that this technique could be further developed by characterizing the effects of siRNAs that target other relevant molecules, such as the mRNA for serotonin receptors, kinases and phosphatases.

### Example 6

### Intermittent (but not Sustained) Hypoxia Increases BDNF in Ventral Cervical Gray Matter

Through a series of experiments, applicants were able to show that intermittent (but not sustained) hypoxia increases BDNF protein concentration in the ventral cervical spinal cord of anesthetized rats (C4-C5), an effect correlated with the magnitude of pLTF. It is unknown if ventilatory LTF in awake rats occurs by similar BDNF-dependent mechanisms, or if sustained hypoxia affects spinal BDNF synthesis.

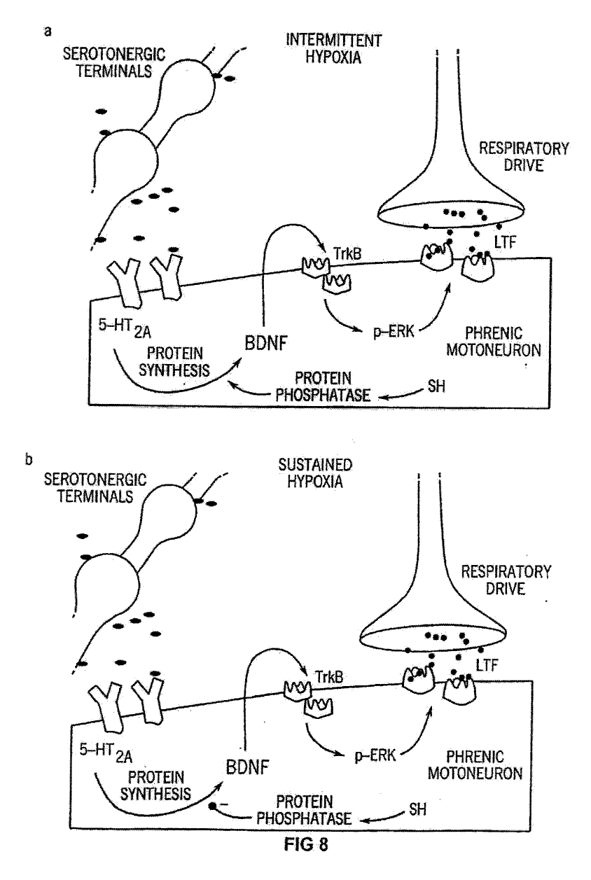

Accordingly, tissues were harvested from three unanesthetized rat groups exposed to varied oxygen environments (n=4, each): 1) control (normoxia only), 2) intermittent hypoxia (5, 5 min hypoxic episodes; 10.5% O₂; 5 min normoxic intervals), and 3) sustained hypoxia (25 min of sustained hypoxia; 10.5% O₂). After the hypoxic exposures, rats were returned to normoxia for 60 min. Following treatments, the C4-C5 ventral gray matter was harvested and assayed for BDNF protein concentration (ELISA; R&D Systems). Similar to anesthetized rats, intermittent hypoxia increased BDNF protein concentration in the ventral gray matter of the cervical spinal cord of awake rats. By contrast, an equivalent duration of sustained hypoxia did not change BDNF protein concentration (see FIG. 8). Thus, similar to pLTF, a protocol known to elicit ventilatory LTF in awake rats, resulted in an increase in ventral cervical BDNF concentration. This effect on BDNF synthesis exhibits similar pattern-sensitivity to phrenic and ventilatory LTF.

### Example 7

### Intermittent (but not Sustained) Hypoxia Elicits Persistent Activation of ERK1/2 MAP Kinases

As indicated hereinabove, ERK 1/2 MAP kinases have many of the requisite characteristics to play a prominent role in pLTF. For example, ERK 1/2 MAP kinases are critically involved in important models of synaptic plasticity. Of particular relevance to pLTF, ERK 1/2 is activated by 5-HT2A receptor activation, an effect associated with reactive oxygen species in some cell types. Similarly, BDNF and TrkB receptor activation rapidly activate ERK 1/2 MAP kinases. Since ERK 1/2 MAP kinases are involved in glutamate receptor trafficking applicants believe that they are logical candidates to translate BDNF signaling into synaptic enhancement, thereby establishing pLTF. Drugs were used that differentially target ERK 1/2 versus other possible MAP kinases such as p38 to demonstrate the feasibility of this idea.

In order to investigate whether intermittent (but not sustained) hypoxia elicits persistent activation of ERK1/2 MAP kinases, ventral cervical (C4-C5) spinal segments were isolated from three groups of awake rats (n=4 per group): 1) normoxia controls, 2) intermittent hypoxia (5,5-min episodes, 10.5%) and 3) sustained hypoxia (25 min, 10.5%). 60-min post-hypoxia, tissues were harvested and stored for immunoblots (upper panel) using antibodies for phospho-ERK 1/2 (Cell Signaling) or total ERK 1/2 (Santa Cruz). The ratio of phosphorylated (activated) ERK1/2 to total ERK 1/2 was significantly increased 60 min post-intermittent (but not sustained) hypoxia (see FIG. 10B). Similar changes were not seen in p38 immunoreactivity (data not shown), an additional member of the MAP kinase family. Thus, ERK 1/2 is activated in a manner consistent with a major role in pLTF.

### Example 8

### ERK1/2 MAP Kinase Activation is BDNF-Dependent

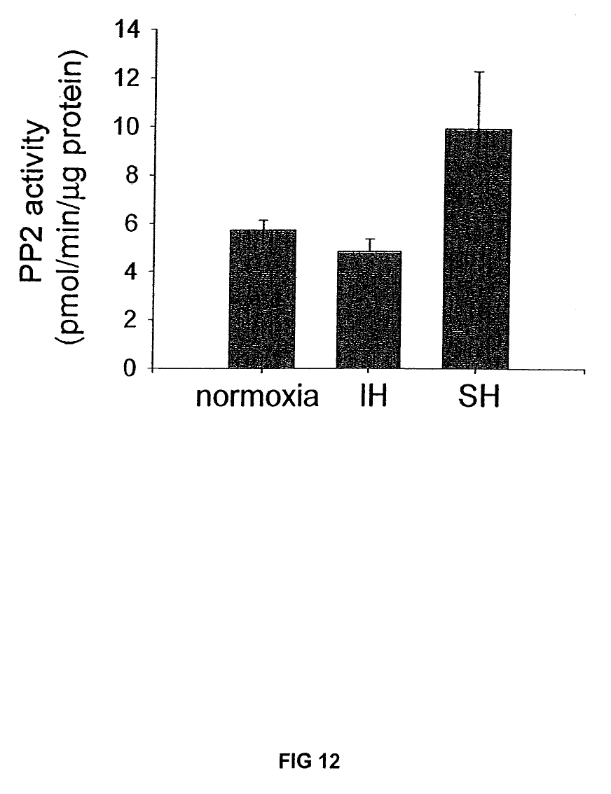

To determine that ERK1/2 MAP kinase activation is BDNF-dependent (downstream from BDNF signaling), BDNF siRNA was injected over C4 in two anesthetized rats: one received normoxia (control), while the other received intermittent hypoxia. This technique effectively prevents hypoxia-induced BDNF synthesis near phrenic motoneurons. Tissues were harvested and ERK 1/2 activation was assessed via immunoblot. These preliminary data indicate that preventing new BDNF synthesis with BDNF siRNA also prevents hypoxia-induced ERK1/2 MAP kinase activation, suggesting that the former causes the latter (see FIG. 12).

### Example 9

### Activation of ERK1/2 MAP Kinase, but not CaMKII, is Necessary for pLTF

To determine if the activation of ERK1/2 MAP kinase alone is necessary for pLTF, one anesthetized rat was pre-treated with intrathecal UO126 (0.4 mg; 100 mM), a MAP kinase kinase inhibitor, 30 min prior to intermittent hypoxia. The inhibition of ERK1/2 MAP kinase activation blocked pLTF. In contrast, blocking activation of an unrelated kinase, CaMKII (KN62 and KN93, n=1 each; 2 mg; 250 mM), did not block pLTF. These results demonstrate the feasibility of targeting ERK 1/2 to modify synaptic function in the spinal cord.

### Example 10

### MAP Kinase Activation Facilitates Phrenic Burst Amplitude

To determine if MAP kinase activation is sufficient to facilitate phrenic burst amplitude, one anesthetized rat was intrathecally injected with anisomycin (100 mg; 20 mM), a protein synthesis inhibitor that also non-specifically activates MAP kinases. Thirty min-post-injection, phrenic burst amplitude was increased by 60% above pre-injection levels. Increased phrenic amplitude following anisomycin is likely due to MAP kinase activation since pretreatment with a selective MAP kinase inhibitor (UO126; 0.4 mg; 100 mM) prevented the effect (n=1). Because anisomycin activates multiple MAP kinases (especially p38) (data not shown). Applicants believe that these preliminary data support the hypothesis that ERK 1/2 MAP kinase activation is sufficient to facilitate phrenic activity similar to pLTF. Thus, by targeting molecules that regulate ERK 1/2 activation, synaptic function in the spinal cord may be modified.

### Example 11

### Sustained (but not Intermittent) Hypoxia Increases Protein Phosphatase (PP) Activity

Three awake rats were given normoxia, intermittent hypoxia (5 episodes of 5 min; 10.5%) or sustained hypoxia (25 min; 10.5%). Ventral C4-C5 was harvested 15 min post-hypoxia and assayed for serine/threonine protein phosphatase activity using a commercially available kit (Promega; Madison, Wis.). Buffer conditions were optimized to preferentially target protein phosphatase 2A (PP2A); however, the effects of protein phosphatases 2B or 2C also need to be accounted for in this assay. The rat exposed to sustained hypoxia had elevated PP2 activity compared to rats exposed to normoxia or intermittent hypoxia; intermittent hypoxia had no effect on PP2 activity. Thus, PP2 activation has the potential to differentiate sustained and intermittent hypoxia in their ability to elicit pLTF (see FIG. 12).

### Example 12

### Sustained Hypoxia Elicits Phrenic LTF Following Protein Phosphatase Inhibition

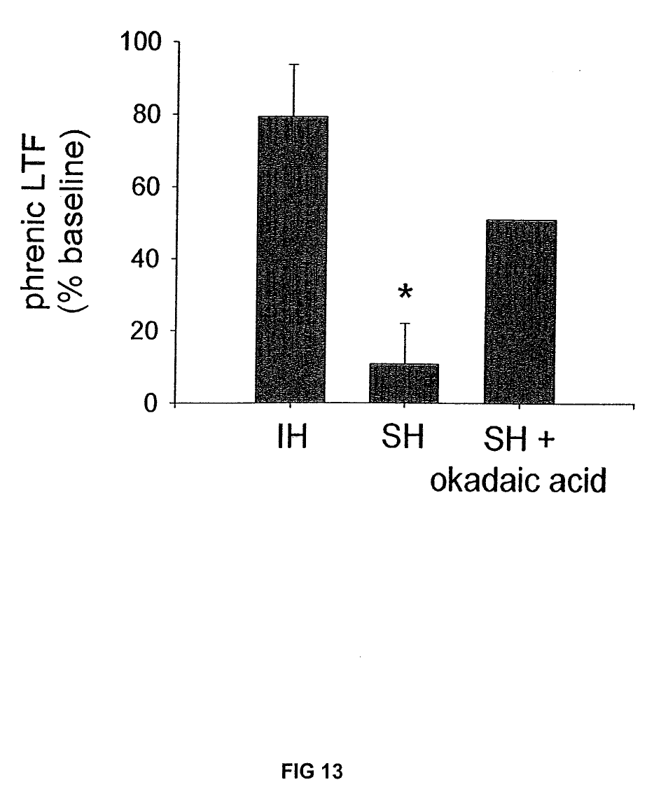

Since preliminary data suggest that sustained hypoxia uniquely increases phosphatase activity, applicants believed that protein phosphatase inhibition removes the inhibitory constraint and reveals pLTF following sustained hypoxia. Accordingly, in one anesthetized rat, 10 ml okadaic acid (0.15 mg; 20 mM), a protein phosphatase 1/2A (PP1 and PP2A) inhibitor, was delivered intrathecally to C4 30 min prior to sustained hypoxia (25 min). 60 min following sustained hypoxia, a modest pLTF was apparent (see FIG. 13). This finding has been successfully repeated in three additional rats. Collectively, these data support the hypothesis that preferential activation of protein phosphatases by sustained hypoxia halts the mechanism leading to pLTF by dephosphorylation at critical steps in the signaling cascade (e.g., PKC; FIG. 8); elimination of that “brake” enables pLTF expression following sustained hypoxia.

### DISCUSSION

In accordance with the scope of the invention, collectively, the above examples demonstrate the respective roles of BDNF, MAP kinases and protein phosphatases in neuronal responses to intermittent hypoxia (and possibly pLTF). Applicants have also demonstrated the differential capacity of intermittent versus sustained hypoxia to elicit long-lasting, functional alterations in spinal motoneurons, and may provide the rationale for the development of new therapeutic approaches for the treatment of devastating respiratory control disorders such as obstructive sleep apnea, Sudden Infant Death Syndrome, respiratory insufficiency following spinal cord injury or during neurodegenerative disease.

In particular, genes such as BDNF and PP2A may be targeted by siRNA molecules through either direct (intrathecal) or indirect (intramuscular) in vivo delivery to affect gene expression and resulting in physiological change. As exemplified above, the invention provides that direct delivery of the siRNA composition into the intrathecal space of a mammal, effectively interfered with BDNF mRNA. The interference with BDNF mRNA blocked increases in BDNF in the cervical spinal cord elicited by a reduced flow of oxygen called intermittent hypoxia, which causes a form of serotonin-dependent spinal synaptic plasticity known as phrenic long-term facilitation (pLTF).

Furthermore, the invention provides that indirect delivery of the BDNF siRNA composition intramuscularly (into muscles innervated by nerve cells) protected siRNA molecules from circulating RNAses in the blood and resulted in transport of intact siRNA molecules back to the nerve cells. This indirect transport of siRNA molecules from the muscles to the nerve cells resulted in blocking LTF following intermittent hypoxia in the hypoglossal nerve (the motor nerve of the tongue) but not phrenic nerve (associated with the diaphragm) motor output, or in the converse depending on where the siRNA had been injected. These findings demonstrate new roles and regulatory mechanisms for BDNF, and illustrate the utility of using siRNA technology to investigate and manipulate gene function in the brain or spinal cord in vivo.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### PUBLICATIONS

1. Mitchell, G. S. & Johnson, S. M. Neuroplasticity in respiratory motor control. J. Appl. Physiol. 94, 358-374 (2003).
2. Feldman, J. L., Mitchell, G. S. & Nattie, E. E. Breathing: Rhythmicity, plasticity and chemosensitivity. Annu. Rev. Neurosci. 26, 239-266 (2003).
3. Baker, T. L., Fuller, D. D., Zabka, A. G. & Mitchell, G. S. Respiratory plasticity: differential actions of continuous and episodic hypoxia and hypercapnia. Respir. Physiol. 129, 25-35 (2001).
4. Mitchell, G. S. et al. Intermittent hypoxia and respiratory plasticity. J. Appl. Physiol. 90, 2466-2475 (2001).
5. Prabhakar, N. R. Oxygen sensing during intermittent hypoxia: cellular and molecular mechanisms. J. Appl. Physiol. 90, 1986-1994 (2001).
6. Fuller, D. D., Zabka, A. G., Baker, T. L. & Mitchell, G. S. Selected Contribution: Phrenic long-term facilitation requires 5-HT receptor activation during but not following episodic hypoxia. J. Appl. Physiol. 90, 2001-2006 (2001).
7. Baker-Herman, T. L. & Mitchell, G. S. Phrenic long-term facilitation requires spinal serotonin receptor activation and protein synthesis. J. Neurosci. 22, 6239-6246 (2002).
8. Thoenen, H. Neurotrophins and neuronal plasticity. Science 270, 593-598 (1995).
9. Thoenen, H. Neurotrophins and activity-dependent plasticity. Prog. Brain Res. 128, 183-191 (2000).
10. Black, I. B. Trophic regulation of synaptic plasticity. J. Neurobiol. 41, 108-118 (1999).
11. Schinder, A. F. & Poo, M-m. The neurotrophin hypothesis for synaptic plasticity. Trends Neurosci. 23, 639-645 (2000).
12. Poo, M-m. Neurotrophins as synaptic modulators. Nature Rev. 2, 1-9 (2001).
13. Bach, K. B. & Mitchell, G. S. Hypercapnia-induced long-term depression of respiratory activity requires a2-adrenergic receptors. J. Appl. Physiol. 84, 2099-2105 (1998).
14. Fire, A. et al. Potent and specific genetic interference by double-stranded RNA in Caenorhabditis elegans. Nature 391, 806-811 (1998).
15. Hammond, S. M., Bernstein, E., Beach, D. & Hannon, G. J. An RNA-directed nuclease mediates post-transcriptional gene silencing in Drosophila cells. Nature 404, 293-296 (2000).
16. Bavis, R. W. & Mitchell, G. S. Selected Contribution: Intermittent hypoxia induces phrenic long-term facilitation in carotid-denervated rats. J. Appl. Physiol. 94, 399-409 (2003).
17. Solomon, I. C. Excitation of phrenic and sympathetic output during acute hypoxia: contribution of medullary oxygen detectors. Respir. Physiol. 121, 101-117 (2000).
18. Bodineau, L. & Larnicol, N. Brainstem and hypothalamic areas activated by tissue hypoxia: Fos-like immunoreactivity induced by carbon monoxide inhalation in the rat. Neuroscience 108, 643-653 (2001).
19. Kokaia, Z. et al. Regional brain-derived neurotrophic factor mRNA and protein levels following transient forebrain ischemia in the rat. Brain Res. Mol. Brain. Res. 38, 139-144 (1996).
20. Blitz, D. M. & Rameriz, J. M. Long-term modulation of respiratory network activity following anoxia in vitro. J. Neurophysiol. 87, 2964-2971 (2002).
21. Patterson, S. L., Grover, L. M., Schwartzkroin, P. A. & Bothwell, M. Neurotrophin expression in rat hippocampal slices: a stimulus paradigm inducing LTP in CA1 evokes increases in BDNF and NT-3 mRNAs. Neuron 9, 1081-1088 (1992).
22. Richerson, G. B., Wang, W., Tiwari, J. & Bradley, S. R. Chemosensitivity of serotonergic neurons in the rostral ventral medulla. Respir. Physiol. 129, 175-190 (2001).
23. Veasey, S. C., Fomal, C. A., Metzler, C. W. & Jacobs, B. L. Response of serotonergic caudal raphe neurons in relation to specific motor activities in freely moving cats. J. Neurosci. 15, 5346-5359 (1995).
24. Uchino, H., Lindvall, O., Siesjo, B. K. & Kokaia, Z. Hyperglycemia and hypercapnia suppress BDNF gene expression in vulnerable regions after transient forebrain ischemia in the rat. J. Cereb. Blood Flow Metab. 17, 1303-1308 (1997).
25. Caplen, N. J., Parrish, S., Imani, F., Fire, A. & Morgan, R. A. Specific inhibition of gene expression by small double-stranded RNAs in invertebrate and vertebrate systems. Proc. Natl. Acad. Sci. USA 98, 9742-9747 (2001).
26. Elbashir, S. M. et al. Duplexes of 21-nucleotide RNAs mediate RNA interference in cultured mammalian cells. Nature 411, 494-498 (2001).
27. Basura, G. J., Zhou, S. Y., Walker, P. D. & Goshgarian, H. G. Distribution of serotonin 2A and 2C receptor mRNA expression in the cervical ventral horn and phrenic motoneurons following spinal cord hemisection. Exp. Neurol. 169, 255-263 (2001).
28. Balkowiec A. & Katz, D. M. Activity-dependent release of endogenous brain-derived neurotrophic factor from primary sensory neurons detected by ELISA in situ. J. Neurosci. 20, 7417-7423 (2000).
29. Tongiorgi, E., Righi, M. & Cattaneo, A. Activity-dependent dendritic targeting of BDNF and TrkB mRNAs in hippocampal neurons. J. Neurosci. 17, 9492-9505 (1997).
30. Li, Y. X., Zhang, Y., Lester, H. A., Schuman, E. M. & Davidson, N. Enhancement of neurotransmitter release induced by brain-derived neurotrophic factor in cultured hippocampal neurons. J. Neurosci. 18, 10231-10240 (1998).
31. Schinder, A. F., Berninger, B. & Poo, M. Postsynaptic target specificity of neurotrophin-induced presynaptic potentiation. Neuron 25, 151-163 (2000).
32. Levine, E. S., Crozier, R. A., Black, I. B. & Plummer, M. R. Brain-derived neurotrophic factor modulates hippocampal synaptic transmission by increasing N-methyl-D-aspartic acid receptor activity. Proc. Natl. Acad. Sci. USA 95, 10235-10239 (1998).
33. Grosshans, D. R., Clayton, D. A., Coultrap, S. J. & Browning, M. D. LTP leads to rapid surface expression of NMDA but not AMPA receptors in adult rat CA1. Nat. Neurosci. 5, 27-33 (2002).
34. Malinow, R. & Malenka, R. C. AMPA receptor trafficking and synaptic plasticity. Annu. Rev. Neurosci. 25, 103-126 (2002).
35. Tanaka, T., Saito, H. & Matsuki, N. Inhibition of GABAA synaptic responses by brain-derived neurotrophic factor (BDNF) in rat hippocampus. J. Neurosci. 17, 2959-2966 (1997).
36. Kafitz, K. W., Rose, C. R., Thoenen, H. & Konnerth, A. Neurotrophin-evoked rapid excitation through TrkB receptors. Nature 401, 918-921 (1999).
37. Ling, L. et al. Chronic intermittent hypoxia elicits serotonin-dependent plasticity in the central neural control of breathing. J. Neurosci. 21, 5381-5388 (2001).
38. Kinkead, R. et al. Cervical dorsal rhizotomy enhances serotonergic innervation of phrenic motoneurons and serotonin-dependent long-term facilitation of phrenic motor output in rats. J. Neurosci. 18, 8436-8443 (1998).
39. Johnson, R. A., Okragly, A. J., Haak-Frendscho, M. & Mitchell, G. S. Cervical dorsal rhizotomy increases brain-derived neurotrophic factor and neurotrophin-3 expression in the ventral spinal cord. J. Neurosci. 20, RC77 1-5 (2000).
40. Kinney, H. C., Filiano, J. J. & White, W. F. Medullary serotonergic network deficiency in the sudden infant death syndrome: review of a 15-year study of a single dataset. J. Neuropathol. Exp. Neurol. 60, 228-247 (2001).
41. Strollo, P. J. & Rogers, R. M. Obstructive sleep apnea. N. Engl. J. Med. 334, 99-104 (1996).
42. Bixler, E. O. et al. Prevalence of sleep-disordered breathing in women: effects of gender. Am. J. Respir. Crit. Care Med. 163, 608-613 (2001).
43. Young, T., Peppard, P. E. & Gottlieb, D. J. Epidemiology of obstructive sleep apnea: a population health perspective. Am. J. Respir. Crit. Care Med. 165, 1217-1239 (2002).
44. Veasey, S. C., Panckeri, K. A., Hoffman, E. A., Pack, A. I. & Hendricks, J. C. The effects of serotonin antagonists in an animal model of sleep-disordered breathing. Am. J. Respir. Crit. Care Med. 153, 776-786 (1996).
45. Zabka, A. G., Behan, M., Mitchell, G. S. Long term facilitation of respiratory motor output decreases with age in male rats. J. Physiol. 531, 509-514 (2001).
46. Jackson, A. B. & Groomes, T. E. Incidence of respiratory complication following spinal cord injury. Arch. Phys. Med. Rehabil. 75, 270-275 (1994).
47. Mansel, J. K. & Norman, J. R. Respiratory complications and management of spinal cord injuries. Chest 97, 1446-1452 (1990).
48. Fuller, D. D., Johnson, S. M., Olson, E. B. Jr. & Mitchell, G. S. Synaptic pathways to phrenic motoneurons are enhanced by chronic intermittent hypoxia following cervical spinal cord injury. J. Neurosci. 23, 2993-3000. (2003).
49. Thoenen, H. & Sendtner, M. Neurotrophins: from enthusiastic expectations through sobering experiences to rational therapeutic approaches. Nat. Neurosci. 5, 1046-1050 (2002).
50. Millhom D E, Eldridge F L, Waldrop T G (1980a). Prolonged stimulation of respiration by a new central neural mechanism. Respir Physiol 41:87-103.
51. Millhom D E, Eldridge F L, Waldrop T G (1980b). Prolonged stimulation of respiration by endogenous central serotonin. Respir Physiol 42:171-88.

We claim:

1. A method of regulating translational activity of a target gene in a nerve cell of the central nervous system of a mammal, the method comprising:
a) providing a small interfering RNA (siRNA) composition constructed to have a strand complementary to a portion of the target gene; and
b) delivering the siRNA composition to a target site on the mammal to cause down-regulation of the target gene in the nerve cell, wherein the target site is a muscle tissue innervated by nerve cell(s) or cerebrospinal space.

2. The method of claim 1 wherein the target gene is brain derived neurotrophic factor (BDNF).

3. The method of claim 1 wherein the target gene comprises a receptor or a signaling molecule downstream of BDNF, acting in concert with BDNF-related molecules or a molecule that regulates BDNF.

4. The method of claim 3 wherein the target gene is a protein phosphatase, preferably PP2A.

5. The method of claim 1 wherein the muscle tissue is a tongue or a diaphragm muscle.

6. The method of claim 1 wherein the muscle tissue is involved in motor behaviors selected from the group consisting of breathing, locomotion, postural control, speech, reaching, grasping and a combination thereof.

7. The method of claim 1 wherein the mammal is a human.

8. The method of claim 1, wherein the siRNA composition is delivered to the target site in the presence of a delivery reagent.

9. The method of claim 8 wherein the delivery reagent is selected from the group consisting of a lipid, a cationic lipid, a phospholipid, and a liposome.

10. The method of claim 8, wherein the delivery reagent is oligofectaminet.

11. The method of claim 1 wherein the nerve cell is a motoneuron that sends processes from the cell body in the medulla or spinal cord to a target muscle or to a sensory neuron that sends processes to the muscle or skin.

12. The method of claim 1 wherein the ability to regulate translational activity enables treatment of a respiratory control disorder selected from the group consisting of obstructive sleep apnea, respiratory insufficiency following spinal cord injury, respiratory insufficiency caused by neurodegenerative motoneuron disease, respiratory deficiency due to polio and sudden infant death syndrome.

13. A method of down-regulating a target gene in a nerve cell of the central nervous system of a mammal, the method comprising:
a) providing a small interfering RNA (siRNA) composition constructed to have a strand complementary to a portion of the target gene; and
b) delivering the siRNA composition to a target site on the mammal to cause down-regulation of the target gene in the nerve cell, wherein the target site is a muscle tissue innervated by nerve cell(s) or cerebrospinal space.

14. The method of claim 13 wherein the target gene is brain derived neurotrophic factor (BDNF).

15. The method of claim 13 wherein the target gene comprises a receptor or a signaling molecule downstream of BDNF, acting in concert with BDNF-related molecules or a molecule that regulates BDNF.

16. The method of claim 15 wherein the target gene is a protein phosphatase, preferably PP2A.

17. The method of claim 13 wherein the muscle tissue is a tongue or a diaphragm muscle.

18. The method of claim 13 wherein the muscle tissue is involved in motor behaviors selected from the group consisting of breathing, locomotion, postural control, speech, reaching, grasping and a combination thereof.

19. The method of claim 13 wherein the mammal is a human.

20. The method of claim 13, wherein the siRNA composition is delivered to the target site in the presence of a delivery reagent.

21. The method of claim 20, wherein the delivery reagent is Oligofectamine™.

22. The method of claim 13 wherein the nerve cell is a motoneuron that sends processes from the cell body in the medulla or spinal cord to a target muscle or to a sensory neuron that sends processes to the muscle or skin.

23. The method of claim 13 wherein the ability to down-regulate target genes in nerve cells enables treatment of a respiratory control disorder selected from the group consisting of obstructive sleep apnea, respiratory insufficiency following spinal cord injury, respiratory insufficiency during neurodegenerative motoneuron disease, respiratory insufficiency following polio, and sudden infant death syndrome.

24. A kit for down regulating a target gene in a nerve cell in the central nervous system of a mammal, the kit comprising:
a) an siRNA composition constructed to have a strand complementary to a portion of the target gene; and
b) instructions for delivering the siRNA composition into a target site on the mammal, such that the target gene in the nerve cell is down-regulated.

25. The kit of claim 24 wherein the target gene is BDNF or a protein phosphatase selected from the group consisting of PP1, PP2A, PP2B, and PP2C.
